(19) United States
(12) Patent Application Publication
(10) Pub. No.: US 2021/0145843 A1
(43) Pub. Date: May 20, 2021

(54) COMPOSITIONS AND METHODS ASSOCIATED WITH HAPTOGLOBIN RELATED PROTEIN

(71) Applicant: SALAQUA DIAGNOSTICS, INC., New York, NY (US)

(72) Inventor: Wayne Comper, N. Ringwood, (AU)

(73) Assignee: SALAQUA DIAGNOSTICS, INC., New York, NY (US)

(21) Appl. No.: 17/135,124

(22) Filed: Dec. 28, 2020

Related U.S. Application Data
(63) Continuation of application No. PCT/US2019/039419, filed on Jun. 27, 2019.
(60) Provisional application No. 62/824,764, filed on Mar. 27, 2019.
(60) Provisional application No. 62/824,423, filed on Mar. 27, 2019.
(60) Provisional application No. 62/824,417, filed on Mar. 27, 2019.
(60) Provisional application No. 62/691,442, filed on Jun. 28, 2018.

(51) Int. Cl. A61K 31/55; G01N 33/566; A61K 38/17; G01N 33/68

(52) U.S. Cl. CPC A61K 31/55; G01N 33/566; G01N 2800/22; G01N 33/6893; A61K 38/17

(57) ABSTRACT

The present disclosure relates to compositions and methods associated with haptoglobin related protein (HRP), including compositions and methods associated with diagnosis and treatment of renal salt wasting (RSW) and the syndrome of inappropriate anti-diuretic hormone secretion (SIADH), as well as diuretic compositions and associated methods.

### CROSS-REFERENCE TO RELATED APPLICATIONS

This application is a continuation of International Patent Application No. PCT/US2019/039419 filed Jun. 27, 2019, which claims priority to U.S. Provisional Application Ser. No. 62/691,442 filed Jun. 28, 2018, U.S. Provisional Application Ser. No. 62/824,417 filed Mar. 27, 2019, U.S. Provisional Application Ser. No. 62/824,423 filed Mar. 27, 2019, and U.S. Provisional Application Ser. No. 62/824,764 filed Mar. 27, 2019, the contents of each of which are incorporated by reference in their entirety.

### SEQUENCE LISTING

The specification further incorporates by reference the Sequence Listing submitted herewith via EFS on Dec. 28, 2020. Pursuant to 37 C.F.R. § 1.52(e)(5), the Sequence Listing text file, identified as 083361_0107_SL.txt is 15,777 bytes in size and was created on Dec. 28, 2020. The entire contents of the Sequence Listing are hereby incorporated by reference. The Sequence Listing does not extend beyond the scope of the specification and thus does not contain new matter.

### FIELD OF THE INVENTION

The field of the invention relates to compositions and methods associated with Haptoglobin related protein (HRP), including compositions and methods associated with diagnosis and treatment of renal salt wasting (RSW) and the syndrome of inappropriate anti-diuretic hormone secretion (SIADH), as well as diuretic compositions and associated methods.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art. Hyponatremia, defined as serum sodium <135 mEq/L, is the most common electrolyte abnormality encountered worldwide and is an independent risk factor for higher morbidity and mortality rates [35,36]. Symptoms related to hyponatremia have been traditionally associated with severe hyponatremia and acute reductions in serum sodium, but there is a growing awareness that even mild hyponatremia is associated with mental dysfunction, unsteady gait, osteoporosis, increased falls and bone fractures [7-9, 37-40]. Based on this awareness, there is an evolving tendency to treat every patient with hyponatremia. This recommendation creates an urgent need to assess with assurance the cause of the hyponatremia in a group of patients with diverse clinical associations and different therapeutic goals.

Two conditions which may cause hyponatremia are renal salt wasting (RSW) (previously known as cerebral salt wasting (CSW)) and the syndrome of inappropriate section of anti-diuretic hormone (SIADH). Patients suffering from RSW can be either normonatremic or hyponatremic and can also have low serum uric acid levels. For example, but not by way of limitation, patients with Alzheimer's or other neurological conditions may present as normonatremic while still suffering from RSW. The combination of low serum uric acid concentrations and defective renal tubular transport for uric acid in these patients results in an increase in the fractional excretion of uric acid (FEUrate). RSW mimics SIADH in many clinical parameters with the important exception that patients suffering from RSW have diminished total body water and sodium. In contrast, total body fluids are increased in SIADH. RSW occurs both as an acute condition, e.g., as observed in fracture, particularly hip fracture, brain injuries such as subarachnoid hemorrhages, and other traumas or as a chronic condition, e.g., as observed in cancer, neurological diseases, and viral or parasitic diseases.

The present volume approach to hyponatremia, which has been in existence for decades, has been inadequate and misleading, in part because of misconceptions that are unsubstantiated by supportive data. It is extremely difficult to accurately assess the volume status of patients that do not suffer from edema, and therefore RSW patients are frequently misdiagnosed as having SIADH. Clarification of the mechanism underlying RSW and its differentiation from SIADH is critical because of opposing therapeutic goals, which are to provide salt and water to a volume depleted patient with RSW and water restrict a water-loaded patient with SIADH.

Differentiating SIADH from RSW has been extremely difficult to accomplish, in part because of significant overlapping clinical findings between both syndromes. Both syndromes are associated with intracranial diseases, have normal renal, thyroid and adrenal function, are hyponatremic and hypouricemic and have concentrated urines, high urine sodium (“UNa”) over 40 mEq/L, and high fractional excretion (FE) of urate. Below is a list of features common to SIADH and RSW, except divergent volume status:

Clinical findings common to both SIADH and RSW
Association with intracranial disease
Hyponatremia
Concentrated urine
Urine sodium [Na] usually > 30 mEq/L
Non-edematous
Hypouricemia, with increased fractional excretion urate [FEurate]
Only difference between SIADH and RSW

| | |
|---|---|
| Volume state: | normal/high in SIADH |
| | low in RSW |

The only clinical difference is the state of their ECV, being euvolemic or hypervolemic in SIADH and hypovolemic in RSW. Again, determining the volume status of non-oedematous patients has been very challenging.

The overlapping of major clinical characteristics between SIADH and RSW and the perception that RSW is a rare clinical entity have virtually eliminated RSW from consideration at the bedside. This diagnostic dilemma needs to be urgently resolved because of the evolving awareness that hyponatremic patients are symptomatic and should therefore be treated [8, 9]. These perceptions and recommendations are in large part influenced by reports of unsteady gait, a fourfold increase in fall rates to be equal between serum sodium of 115 to 132 mEq/L, fourfold increase in bone fractures in elderly hyponatremic patients and increasing osteoporosis with chronic hyponatremia [8, 9,41].

There is universal agreement that extracellular volume cannot be assessed with any degree of accuracy by usual clinical criteria, yet the approach to hyponatremia starts with an assessment of volume. The ineffectiveness of this volume approach is becoming even more evident by an objective review of the literature and recent publications of RSW occurring in patients without clinical cerebral disease [4, 5]. 83 to 94% of hyponatremic patients with different forms of neurosurgical diseases have been reported to have hypovolemia with high UNa that met the criteria for RSW as compared to hypervolemic patients with SIADH.

It is clear that the prevalence of RSW (non-cerebral disease form) is now recognized to be far more common than previously thought and is comparable to the incidence of SIADH amongst hyponatremic patients [42]. Moreover, water restricting these patients for an erroneous diagnosis of SIADH has been reported to increase morbidity and mortality rates in patient with subarachnoid hemorrhage and by others [4, 6, 19]. Previous attempts to address the issue of misdiagnosis of RWS as SIADH include the development of an algorithm that utilizes FEurate as a pivotal determination. This algorithm eliminates the determination of plasma renin, aldosterone and A/BNP and UNa, which have been found to be ineffective and often misleading.

The urgency in resolving the diagnostic and therapeutic dilemma is important because of divergent therapeutic goals of appropriately water restricting those with SIADH and increasing salt and water with RSW to avoid iatrogenic increases in morbidity and mortality. The recent recommendations to treat most or all patients with hyponatremia introduce an urgency to resolve this diagnostic and therapeutic dilemma. Accordingly, there remains a need to develop methods which accurately and rapidly distinguish hyponatremia associated RSW from that associated with SIADH in an individual.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In certain embodiments, there is provided a method for treating an individual for RSW or for one or more symptoms thereof comprising: providing or having provided a control describing the amount of plasma or serum HPR in a normal individual; assessing or having assessed a plasma or serum test sample obtained from an individual for whom RSW or one or more symptoms thereof is to be treated, to determine the amount of plasma or serum HPR contained in the test sample; comparing or having compared the amount of plasma or serum HPR in the test sample with the control; salt and water-treating the individual, or treating the individual with an HPR antagonist, where the amount of plasma or serum HPR in the test sample is greater than the amount of plasma or serum HPR in the control; thereby treating the individual for RSW or one or more symptoms thereof.

In certain embodiments, there is provided a method for treating an individual for RSW, or for one or more symptoms thereof comprising: providing, or having provided, a control describing the amount of plasma or serum HPR in the serum or plasma of a normal individual; assessing, or having assessed, a plasma or serum test sample obtained from an individual for whom RSW, or one or more symptoms thereof is to be treated, to determine the amount of plasma or serum HPR comprised in the test sample; comparing, or having compared, the amount of plasma or serum HPR in the test sample with the control to determine whether the individual has an amount of plasma or serum HPR that is more than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is less than or equal to the amount of plasma or serum HPR in the control, not treating the individual with salt and water, or not treating the individual with an HPR antagonist, thereby treating the individual for RSW, or for one or more symptoms thereof.

In certain embodiments, there is provided a method of treating an individual suffering from RSW, the method comprising the steps of: providing, or having provided, a control describing the amount of plasma or serum HPR in a normal individual; assessing, or having assessed, a plasma or serum test sample obtained from an individual suffering from RSW, to determine the amount of plasma or serum HPR comprised in the test sample; comparing, or having compared, the amount of plasma or serum HPR in the test sample with the control to determine whether the individual has an amount of plasma or serum HPR that is more than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is less than or equal to the amount of plasma or serum HPR in the control, not treating the individual with salt and water, or not treating the individual with an HPR antagonist, wherein a risk of prolonged RSW in an individual having an amount of plasma or serum HPR that is more than the control is lower following salt and water administration, or following HPR antagonist administration, than is the risk in an individual having an amount of plasma or serum HPR that is the same as or lower than the control following salt and water administration, or HPR antagonist administration; thereby treating an individual suffering from RSW.

In certain embodiments, there is provided a method for treating an individual having symptoms of RSW to minimize said symptoms, the method comprising the following steps: providing, or having provided, a control describing the amount of plasma or serum HPR in a normal individual; assessing, or having assessed, a serum or plasma test sample obtained from an individual for whom one or more symptoms of RSW are to be minimized, to determine the amount of plasma or serum HPR comprised in the test sample; comparing, or having compared, the amount of plasma or serum HPR in the serum or plasma test sample with the control to determine whether the individual has an amount of plasma or serum HPR that is more than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is less than or equal to the amount of plasma or serum HPR in the control, not treating the individual with salt and water, or not treating the individual with an HPR antagonist, wherein a risk of lesser minimization of symptoms of RSW in an individual having an amount of plasma or serum HPR that is more than the control is lower following salt and water administration, or following HPR antagonist administration, than is the risk of lesser minimization of symptoms of RSW in an individual having an amount of plasma or serum HPR that is the same as or less than the control following salt and water administration or following HPR antagonist administration; thereby treating an individual having symptoms of RSW to minimize said symptoms.

In certain embodiments, there is provided a kit for determining whether an individual has RSW, or for use in providing treatment for an individual having RSW or one or more symptoms thereof comprising: a reagent, e.g., an HPR selective agent, for determining the amount of plasma or serum HPR in a plasma or serum test sample obtained from an individual for whom the presence of RSW is to be determined, or for whom treatment is to be provided; written instructions for use in a method described above.

In certain embodiments, there is provided a composition comprising a salt and water, or comprising an HPR antagonist for use in minimizing RSW for one or more symptoms of RSW in an individual wherein: the amount of plasma or serum HPR in a plasma or serum test sample obtained from an individual having RSW or one or more symptoms of RSW relative to the amount of plasma or serum HPR in a normal control describing the amount of plasma or serum HPR in a normal individual is determined; and wherein salt and water, or wherein an HPR antagonist is utilized to treat the individual where the amount of plasma or serum HPR in the plasma or serum test sample is more than the normal control, thereby minimizing RSW one or more symptoms of hyponatremia in an individual.

In certain embodiments, there is provided an HPR antagonist or pharmaceutical composition comprising same for use in treatment of RSW.

In certain embodiments, there is provided a method for determining whether an individual has RSW comprising: determining the amount of plasma or serum HPR in a test sample obtained from an individual having RSW, or having one or more symptoms of RSW, relative to the amount of plasma or serum HPR in a normal control describing the amount of plasma or serum HPR in a normal individual, wherein an amount of plasma or serum HPR in the test sample that is more than the normal control determines that the individual has RSW.

In certain embodiments, there is provided a pharmaceutical composition for treatment of RSW or one or more symptoms thereof in an individual comprising a salt and water, or comprising an HPR antagonist, wherein: the amount of plasma or serum HPR in a test sample obtained from an individual having RSW, or having one or more symptoms of RSW, relative to the amount of plasma or serum HPR in a normal control describing the amount of plasma or serum HPR in a normal individual is determined; and wherein salt and water, or wherein an HPR antagonist, is administered to the individual where the amount of plasma or serum HPR in the test sample is more than the normal control, thereby treating the individual for RSW or one or more symptoms thereof.

In certain embodiments, there is provided a pharmaceutical composition for treatment of RSW comprising an HPR antagonist.

In certain embodiments, there is provided an HPR selective agent for use in determining whether an individual has RSW syndrome comprising: utilizing an HPR selective agent to determine the amount of plasma or serum HPR in a test sample obtained from an individual having one or more symptoms of RSW relative to the amount of plasma or serum HPR in a normal control describing the amount of plasma or serum HPR in normal individual; wherein an amount of plasma or serum HPR in the test sample that is more than the normal control determines that the individual has RSW.

In certain embodiments, the methods disclosed herein comprise determining the amount of plasma or serum HPR related peptide in a test sample, instead of determining the amount of plasma or serum HPR in the test sample. In these embodiments, the control describing the amount of plasma or serum HPR in a normal individual can be used as a comparison.

In certain embodiments, there is provided a pharmaceutical composition including a pharmaceutically effective amount of an HPR antagonist.

In certain embodiments, there is provided a method for treating an individual for SIADH, or for one or more symptoms thereof comprising: providing or having provided a control describing the amount of plasma or serum HPR in a normal individual; assessing or having assessed a plasma or serum test sample obtained from an individual for whom hyponatremia or one more symptoms thereof is to be treated, to determine the amount of plasma or serum HPR comprised in the test sample; comparing or having compared the amount of plasma or serum HPR in the test sample with the control; treating the individual with a vasopressin receptor antagonist where the amount of plasma or serum HPR in the test sample is the same as or less than the amount of plasma or serum HPR in the control; thereby treating the individual for SIADH or one or more symptoms thereof.

In certain embodiments, there is provided a method for treating an individual for SIADH, or for one or more symptoms thereof comprising: providing, or having provided, a control describing the amount of plasma or serum HPR in a normal individual; assessing, or having assessed, a plasma or serum test sample obtained from an individual for whom SIADH, or one or more symptoms thereof is to be treated, to determine the amount of plasma or serum HPR comprised in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is the greater than the amount of plasma or serum HPR in the control, not treating the individual with a vasopressin receptor antagonist, thereby treating the individual for SIADH, or for one or more symptoms thereof.

In certain embodiments, there is provided a method of treating an individual suffering from SIADH, the method comprising the steps of: providing, or having provided, a control describing the amount of plasma or serum HPR in a normal individual; assessing, or having assessed, a plasma or serum test sample obtained from an individual suffering from SIADH, to determine the amount of plasma or serum HPR comprised in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is greater than the amount of plasma or serum HPR in the control, not treating the individual with a vasopressin receptor antagonist, wherein a risk of prolonged SIADH in an individual having an amount of plasma or serum HPR that is the same as or less than the control is lower following vasopressin receptor antagonist administration, than is the risk in an individual having a greater amount of plasma or serum HPR than the control following vasopressin receptor antagonist administration; thereby treating an individual suffering from SIADH.

In certain embodiments, there is provided a method for treating an individual having symptoms of SIADH to minimize said symptoms, the method comprising the following steps: providing, or having provided, a control describing the amount of plasma or serum HPR in a normal individual; assessing, or having assessed, a plasma or serum test sample obtained from an individual for whom one or more symptoms of SIADH are to be minimized, to determine the amount of plasma or serum HPR comprised in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is greater than the amount of plasma or serum HPR in the control, not treating the individual with a vasopressin antagonist, wherein a risk of lesser minimization of symptoms of SIADH in an individual having an amount of plasma or serum HPR that is the same as or less than the control is lower following vasopressin receptor antagonist administration, than is the risk of lesser minimization of symptoms of SIADH in an individual having a greater amount of plasma or serum HPR than the control following vasopressin receptor antagonist administration; thereby treating an individual having symptoms of SIADH to minimize said symptoms.

In certain embodiments, there is provided a kit for determining whether an individual has SIADH or other form of euvolemic hyponatremia, or for use in providing treatment for an individual having SIADH or one or more symptoms thereof comprising: a reagent, e.g., an HPR selective agent, for determining the amount of plasma or serum HPR in a test sample obtained from an individual for whom the presence of SIADH or other form of euvolemic hyponatremia is to be determined, or for whom treatment is to be provided; written instructions for use in a method described above.

In certain embodiments, there is provided a composition comprising a vasopressin receptor antagonist, for use in minimizing SIADH or one or more symptoms of SIADH in an individual wherein: the amount of plasma or serum HPR in a test sample obtained from an individual having SIADH or one or more symptoms of SIADH relative to the amount of plasma or serum HPR in a normal control describing the amount of plasma or serum HPR in a normal individual is determined; and wherein vasopressin receptor antagonist is utilized to treat the individual where the amount of plasma or serum HPR in the test sample is the same as or less than the normal control; thereby minimizing SIADH one or more symptoms of SIADH in an individual.

In certain embodiments, there is provided a method for determining whether an individual has SIADH or other form of euvolemic hyponatremia comprising: determining the amount of plasma or serum HPR in a test sample obtained from an individual having SIADH, or having one or more symptoms of SIADH, relative to the amount of plasma or serum HPR in a normal control describing the amount of plasma or serum HPR in a normal individual, wherein an amount of plasma or serum HPR in the test sample that is the same as or less than the normal control determines that the individual has SIADH or other form of euvolemic hyponatremia.

In certain embodiments, there is provided a pharmaceutical composition for treatment of SIADH or one or more symptoms thereof in an individual comprising a vasopressin receptor antagonist, wherein: the amount of plasma or serum HPR in a test sample obtained from an individual having SIADH, or having one or more symptoms of SIADH, relative to the amount of plasma or serum HPR in a normal control describing the amount of plasma or serum HPR in a normal individual is determined; and wherein vasopressin receptor antagonist, is administered to the individual where the amount of plasma or serum HPR in the test sample is the same as or less than the normal control, thereby treating the individual for SIADH or one or more symptoms thereof.

In certain embodiments, there is provided an HPR selective agent for use in determining whether an individual has SIADH or other form of euvolemic hyponatremia comprising: utilizing an HPR selective agent to determine the amount of plasma or serum HPR in a test sample obtained from an individual having one or more symptoms of SIADH relative to the amount of plasma or serum HPR in a normal control describing the amount of plasma or serum HPR in normal individual; wherein an amount of plasma or serum HPR in the test sample that is the same as or less than the normal control determines that the individual has SIADH or other form of euvolemic hyponatremia.

In certain embodiments, the individual that is the subject of the treatment has an unknown volume status.

In certain embodiments, the individual has chronic neurodegenerative disorder, e.g., Alzheimer's disease, or an acute neurological disease, e.g., sub-arachnoid hemorrhage, or a bone fracture, e.g., a hip fracture.

In certain embodiments, if a test sample from an individual comprises an HPR signal peptide deletion variant, or an amount of plasma or serum HPR signal peptide deletion variant that is less than or equal to the control, the individual is subjected to therapy for euvolemic hyponatremia including, for example, water restriction.

In certain embodiments, there is provided a method for determining the likelihood of an individual developing RSW comprising: assessing the amount of haptoglobin in a test sample obtained from a hyponatremic individual having one or more symptoms of hyponatremia for whom likelihood of development of RSW is to be determined; determining a high likelihood of the individual developing RSW where the individual has an amount of haptoglobin relative to a control describing the amount of haptoglobin in a normal individual that is more than the control; determining a low likelihood of the individual developing RSW where the individual has an amount of haptoglobin relative to a control describing the amount of haptoglobin in a normal individual that is the same as or less than the control, thereby determining the likelihood of an hyponatremic individual developing RSW. In certain embodiments, the individual has a higher risk for developing RSW where the blood concentration of haptoglobin is >200 to 300 mg/dL.

In certain embodiments, there is provided a method for inducing diuresis in an individual comprising administering HPR, or an HPR signal peptide deletion variant, to an individual in whom diuresis is to be induced, thereby inducing diuresis in the individual. In certain embodiments, there is provided a use of HPR, or an HPR signal peptide deletion variant, for inducing diuresis in an individual. In certain embodiments, there is provided a use of HPR, or an HPR signal peptide deletion variant, in the manufacture of a medicament for inducing diuresis in an individual. In certain embodiments, there is provided HPR, or an HPR signal peptide deletion variant, for use in inducing diuresis in an individual.

In certain embodiments, there is provided a method for increasing the fractional excretion of sodium (FENa) in an individual comprising administering HPR, or an HPR signal peptide deletion variant, to an individual in whom FENa is to be increased, thereby increasing FENa in the individual. In certain embodiments, there is provided a use of HPR, or an HPR signal peptide deletion variant, for increasing FENa in an individual. In certain embodiments, there is provided a use of HPR, or an HPR signal peptide deletion variant, in the manufacture of a medicament for increasing FENa in an individual. In certain embodiments, there is provided HPR, or an HPR signal peptide deletion variant, for use in increasing FENa in an individual.

In certain embodiments, there is provided a method for increasing urine flow rate in an individual comprising administering HPR, or an HPR signal peptide deletion variant, to an individual in whom urine flow rate is to be increased, thereby increasing urine flow rate in the individual. In certain embodiments, there is provided a use of HPR, or an HPR signal peptide deletion variant, for increasing urine flow rate in an individual. In certain embodiments, there is provided a use of HPR, or an HPR signal peptide deletion variant, in the manufacture of a medicament for increasing urine flow rate in an individual. In certain embodiments, there is provided HPR, or an HPR signal peptide deletion variant, for use in increasing urine flow rate in an individual.

In certain embodiments, and as described further herein, the individual treated with HPR, or an HPR signal peptide deletion variant, may be oedemic and have one or more associated conditions including congestive heart failure. Further, the individual may be the subject of ongoing therapy including diuretic or anti-hypertensive therapy. In certain embodiments, the individual may be resistant to, or refractory for oedema or diuretic therapy.

In certain embodiments, and as further described herein, the HPR for use in the above described embodiments may lack the canonical HPR signal peptide or leader peptide.

In certain embodiments, there is provided a composition suitable for intravenous administration comprising: HPR, or an HPR signal peptide deletion variant, as an active principle, e.g., as an active principle for inducing diuresis, or for increasing FENa, or for increasing FELi, or for increasing urine flow rate; and a carrier, excipient or solvent suitable for intravenous administration.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Examples of the variation of FENa (log scale) as measured in control rat as a function of time when perfused with serum from either SAH patient (closed circles) or AD patient (open circles). The black line represents the mean values for controls (n=13). The time represents the start of the 30 min collection (Table 2).

FIG. 2. The clinical course of the patient after sustaining SAH is typical of RSW, with excretion of large volumes of urine that required increased volumes of saline to maintain hemodynamic stability: I=input, O=output.

FIG. 3. Recombinant HPR (molecular weight 36.8 kDa) without signal peptide was produced by E coli was >85% purity according to the manufacturer (Origene Technologies, Rockville, USA) and supplied as 150 μg/ml stock solution. For the bolus rat infusion studies a 1 ml bolus was infused as distinct for the studies with clinical plasma samples described in Table 2 where 0.5 ml was infused. A direct comparison of the results in this figure as compared to the results in Table 2 would have to take these different bolus infusion volumes into account. In fact, since only 0.5 ml was used in Table 2 then those results were being generated by only at best 50% of the mass of those in this Figure. Therefore, if the critical point of FIG. 3 is ˜75 μg/ml then this would translate to a concentration of 150 ug/ml for the clinical samples which is in approximate accord with the results of Table 5. Open circles correspond to FENa with negative SD. Filled circles correspond to UFR with positive SD.

### DETAILED DESCRIPTION OF THE EMBODIMENTS

As described herein, the inventors have determined the identity of a natriuretic factor that is upregulated in individuals having acute and chronic disease with known comorbidity with hyponatremia. Further, the inventors show that the factor induces renal salt wasting in a rodent model. These findings are significant and enable one to discriminate RSW from SIADH or other forms of non-oedematous hyponatremia and consequently, to provide an appropriate therapeutic regimen for treatment of RSW.

Moreover, with a quantitative or qualitative assessment of a test sample from a non-oedematous hyponatremic individual for Haptoglobin Related Protein (HPR) or fragment thereof it is possible to determine whether a hyponatremic individual has, or is at risk of developing syndrome of inappropriate anti-diuretic hormone secretion (SIADH) or other form of euvolemic hyponatremia, and therefore, to appropriately select non-oedematous hyponatremic individuals for vasopressin receptor antagonist treatment, and to treat those individuals with vasopressin receptor antagonists. The risk profile for SIADH or other form of euvolemic hyponatremia may be determined without assessment of volume status (i.e., whether hypervolemic or euvolemic) thereby enabling a more targeted and earlier vasopressin receptor antagonist therapy in individuals at risk for SIADH or other form of euvolemic hyponatremia. The present disclosure is of particular use where it is difficult to accurately assess volume status of the individual, and/or an individual presents with symptoms or characteristics that are common to SIADH and other forms of non-oedematous hyponatremia

### 1. Definitions

‘Hyponatremia’ generally refers to a serum concentration of <135 mEq/L. A ‘Hyponatremic’ person is an individual with hyponatremia.

‘Normonatremia’ generally refers to a serum concentration of 135 to 145 mEq/L. A ‘normonatremic’ person is an individual with normonatremia.

‘Hypouricemia’ generally refers to a serum concentration of uric acid of <4 mg/dL. A ‘hypouricemic’ person is generally an individual with hypouricemia.

‘Normouricemia’ generally refers to a serum concentration of uric acid of about 1.9 to 8 mg/dL, typically 2.5 mg/dL to 8 mg/dL in men and 1.9 mg/dL to 7.5 mg/dL in women. A ‘normouricemic’ person generally refers to a person with normauricemia.

‘Hypovolemia’ as used herein generally refers to loss of vascular volume, typically as a result of decreased renal reabsorption of sodium and/or water. As used herein ‘hypovolemia’ is distinguished from dehydration which is an excessive loss of body water, generally from vomiting, diarrhea or sweating. Hypovolemia may include up to 30% loss of volume (for example about 1500 ml) and may be associated with slight increase in diastolic blood pressure and slight decrease in systolic blood pressure. A ‘hypovolemic’ person generally refers to a person who has hypovolemia. The person may also be referred to as ‘volume depleted’.

‘Hypervolemia’ generally refers to an increased vascular volume which may arise from increased renal uptake of salt and/or water, or in oedematous states such as in heart failure, cirrhosis or nephrosis. Hypervolemia may be oedematous or non-oedematous. A person who is ‘hypervolemic’ generally has hypervolemia, generally arising for example from decreased diuresis.

‘Euvolemia’ generally refers to a normal vascular volume. A person who is ‘euvolemic’ generally has a normal volume.

‘An individual having an unknown volume status’ generally refers to an individual for whom vascular volume is unknown. For example, in certain embodiments, it is not known whether the individual is hypovolemic, hypervolemic or euvolemic, e.g., at the time of application of the treatment methods described herein. In certain embodiments, it is not known whether the individual is hypovolemic rather than euvolemic, or vice versa.

‘Fractional excretion of urate’ determines the percent excretion of uric acid that is filtered or presented to the kidneys, normal being between 4 and 11%.

‘Concentrated urine’ is determined by the amount of solute in a given volume, referred to as osmolality (osm) that is greater in urine than in a coexisting serum osmolality, often referred to as Uosm>Posm.

‘Symptom of hyponatremia’ generally refers to one of unsteadiness, weakness, nausea, malaise, lethargy, confusion, decrease mental capacity or cognitive function, decreased level of consciousness, headache, seizures and coma.

‘Symptom of renal salt wasting’ generally refers to unsteadiness, weakness, nausea, malaise, lethargy, confusion, decrease mental capacity or cognitive function, decreased level of consciousness, headache, seizures and coma. Typically, a person in whom symptoms of renal salt wasting are to be minimized has RSW, although generally at the time that the treatment methods described herein are applied to the individual it is not known whether the individual has RSW.

‘Normal individual’ is generally an individual who is normouricemic, normonatremic, without oedema with normal FEurate.

‘Haptoglobin related protein (HPR)’ is a serum protein that exists as a heterodimer of a and p subunits that arise from cleavage of a peptide translated from the HPR gene. The peptide translated from the HPR gene is shown in SEQ ID No: 1, which is the canonical sequence for HPR. The HPR a subunit is about 13.5 kD and contains the amino acid sequence shown in SEQ ID No: 2 and the HPR subunit is about 36.5 kD and contains the amino acid sequence shown in SEQ ID No: 3 [43]. Sequence variants that contain 1 to 2 amino acid differences from the HPR canonical sequence of SEQ ID No: 1 have been observed.

‘Haptoglobin related protein fragments’ and ‘HPR fragments’ as used herein generally refer to any HPR protein lacking one or more amino acids of the full-length HPR protein sequence.

‘HPR signal peptide deletion variant’ generally refers to an HPR peptide sequence or fragment thereof, wherein said HPR or fragment thereof does not comprise all or part of the HPR signal peptide sequence. The HPR signal peptide deletion variant may comprise a part of the canonical HPR signal peptide sequence or none of said sequence. It does not have all of the signal peptide sequence. The signal peptide sequence for HPR is generally 18 amino acids shown in SEQ ID No: 7. The HPR signal peptide deletion variant can be selectively detected by utilizing reagents that bind to the HPR signal peptide deletion variant that do not bind to HPR, or by using reagents that bind to HPR but do not bind to the HPR signal peptide deletion variant.

‘Determining the amount of serum or plasma HPR’ as used herein generally refers to a determination of the concentration of the circulating pool of HPR protein, including full-length HPR, HPR fragments, HPR signal peptide deletion variants, HPR bound to plasma components, e.g. lipids, and multimers of HPR, e.g., dimers and higher-order aggregates of HPR.

‘HPR Control’ as used herein can refer to the amount of HRP determined in sample from a healthy, e.g., non-RSW and non-SIADH, individual. In general, a healthy, e.g., non-RSW and non-SIADH, HRP control value will be about 35 to about 80 μg/ml, about 35 to about 70 μg/ml, about 35 to about 60 μg/ml, about 35 to about 50 μg/ml, about 35 to about 45 μg/ml, or about 40 μg/ml.

‘SIADH’ or ‘syndrome of inappropriate anti-diuretic hormone secretion’ generally refers to a condition in which an excessive and inappropriate increase in antidiuretic hormone gives rise to retention of water and low serum sodium concentration when the subject is ingesting a mandatory adequate amount of water.

‘Renal salt wasting’ syndrome or ‘RSW’ generally refers to a condition in which either excessive loss of salt, or failure to reabsorb salt from renal tissue leads to a low serum sodium concentration and hypovolemia if the subject is ingesting more water than salt but can also occur with normonatremia. Renal salt wasting has previously been referred to as ‘cerebral salt wasting’ or ‘CSW’. Renal salt wasting may be distinguished from SIADH on the basis of the persistently increased FEUA which is observed after correcting the hyponatremia by any means in RSW, but not SIADH and has reduced blood volume as compared to increased blood volume in SIADH.

‘HPR related peptide’ generally refers to a peptide that has amino acid sequence that distinguish the peptide as arising from HPR instead of some other polypeptide, such as Haptoglobin. Exemplary peptides that are unique to HPR and not found in haptoglobin can be determined by alignment of HPR amino acid sequence with the haptoglobin amino acid sequence, not including alignment of leader sequences. See for example Maeda, N. 1985 J. Biol Chem. 11: 6698-6709.

A ‘HPR antagonist’ is a compound or molecule that binds HPR and/or competes with HPR for engagement with proximal tubule cells.

‘HPR selective agent’ is generally a molecule or compound that binds to HPR or a fragment thereof, but not to other molecules or compounds, for example, not to Haptoglobin, thereby enabling detection of HPR or a fragment thereof. An HPR signal peptide deletion variant selective agent that selectively binds to an HPR signal peptide deletion variant does not bind to an HPR that comprises the signal peptide sequence.

‘Inducing diuresis’ generally refers to increasing urine production. This may include increasing FENa, FELi or urinary flow above normal.

‘Fractional excretion of sodium’ (or FENa) determines the percent excretion of sodium that is filtered or presented to the kidneys, normal being 0.2-0.4%.

‘Fractional excretion of lithium’ (or FELi) determines the percent excretion of uric acid that is filtered or presented to the kidneys, normal being about 40%.

‘Urinary flow rate’ generally refers to the volume of urine produced per given time period, normal being 1 to 2 liters per day.

‘Increasing FENa’ generally refers to increasing FENa above normal FENa.

‘Increasing FELi’ generally refers to increasing FELi above normal FELi.

‘Increasing urinary flow rate’ generally refers to increasing urinary flow rate above normal urinary flow rate.

“Comprise” and variations of the term, such as “comprising”, “comprises” and “comprised”, are not intended to exclude further additives, components, integers or steps.

### 2. Treatment and Diagnosis Based on HPR Concentration

As described herein, a particular advantage of the present disclosure is to be able to determine whether an individual's hyponatremia is associated with RSW or SIADH. In certain embodiments, the assessment may be made on the basis of the amount of plasma or serum HPR in a test sample obtained from a hyponatremic or normonatrimic individual.

### 2.1 Methods of Treatment Based on Amount of HPR

In certain embodiments, there is provided a method for treating an individual for RSW, or for one or more symptoms thereof comprising: providing, or having provided, a control describing the amount of plasma or serum HPR in a normal individual; assessing, or having assessed, a test sample obtained from an individual for whom RSW, or one or more symptoms thereof is to be treated, to determine the amount of plasma or serum HPR contained in the test sample; comparing, or having compared, the amount of plasma or serum HPR in the test sample with the control to determine whether the individual has an amount of plasma or serum HPR that is more than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is the same as or less than the amount of plasma or serum HPR in the control, not treating the individual with salt and water, or not treating the individual with an HPR antagonist, thereby treating the individual for RSW, or for one or more symptoms thereof.

In certain embodiments, there is provided a method of treating an individual suffering from RSW, the method comprising the steps of: providing, or having provided, a control describing the amount of plasma or serum HPR in a normal individual; assessing, or having assessed, a test sample obtained from an individual suffering from hyponatremia, to determine the amount of plasma or serum HPR contained in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is the same as or less than the amount of plasma or serum HPR in the control, not treating the individual with salt and water, or not treating the individual with an HPR antagonist, wherein a risk of prolonged RSW in an individual having an amount of plasma or serum HPR that is more than the control is lower following salt and water administration, or following HPR antagonist administration, than is the risk in an individual having an amount of plasma or serum HPR that is the same or less than the control following salt or water administration, or following HPR antagonist administration; thereby treating an individual suffering from RSW.

In certain embodiments, there is provided a method for treating an individual having symptoms of RSW to minimize said symptoms, the method comprising the following steps: providing, or having provided, a control describing the amount of plasma or serum HPR in a normal individual; assessing, or having assessed, a test sample obtained from an individual for whom one or more symptoms of RSW are to be minimized, to determine the amount of plasma or serum HPR contained in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is more than the amount of plasma or serum HPR in the control, treating the individual with salt and water, or treating the individual with an HPR antagonist and; if the amount of plasma or serum HPR in the test sample is the same as or less than the amount of plasma or serum HPR in the control, not treating the individual with salt and water, or not treating the individual with an HPR antagonist, wherein a risk of lesser minimization of symptoms of RSW in an individual having an amount of plasma or serum HPR that is more than the control is lower following salt and water administration, or following HPR antagonist administration, than is the risk of lesser minimization of symptoms of RSW in an individual having an amount of plasma or serum HPR that is the same as or less than the control following salt and water administration, or following HPR antagonist administration; thereby treating an individual having symptoms of RSW to minimize said symptoms.

In certain of the above-described embodiments, if the amount of plasma or serum HPR in the test sample is the same as or less than the amount of plasma or serum HPR in the control, or the amount of plasma or serum HPR related peptide in the test sample is the same as or less than the HPR related peptide in the control, or the amount HPR signal peptide deletion variant is the same as or less than the amount of plasma or serum HPR signal peptide deletion variant in the control, then the individual is more likely to have SIADH or euvolemic hyponatremia and is subjected to therapy for SIADH or other euvolemic hyponatremia including, for example, water restriction. In certain embodiments, individual treated according to the above-described embodiments has an unknown volume status. In certain embodiments, the individual is non-oedematous.

In certain embodiments, there is provided a method for treating an individual for RSW, or for one or more symptoms thereof, e.g., an individual having an unknown volume status, comprising: providing or having provided a control describing the amount of plasma or serum HPR in a normal individual; assessing or having assessed a test sample obtained from an individual for whom RSW or one more symptoms thereof is to be treated, to determine the amount of plasma or serum HPR contained in the test sample; comparing or having compared the amount of plasma or serum HPR in the test sample with the control; treating the individual with a salt and water, or treating the individual with an HPR antagonist where the amount of plasma or serum HPR in the test sample is more than the amount of plasma or serum HPR in the control; thereby treating the individual for RSW or one or more symptoms thereof.

In certain embodiments, there is provided a composition comprising a salt and water, or comprising an HPR antagonist, for use in minimizing RSW or one or more symptoms of hyponatremia in an individual, e.g., where the individual has an unknown volume status wherein: the amount of plasma or serum HPR in a test sample obtained from an individual having RSW or one or more symptoms of RSW relative to the amount of plasma or serum HPR in a normal control describing the amount of plasma or serum HPR in a normal individual is determined; and wherein salt and water, or wherein an HPR antagonist is utilized to treat the individual where the amount of plasma or serum HPR in the test sample is more than the normal control, thereby minimizing RSW, or one or more symptoms of RSW in an individual. In certain embodiments, the test sample is obtained from plasma or urine, although as described herein the test sample may be obtained from other body fluids or tissues. The control may describe an amount of plasma or serum HPR of about 40 μg/ml of plasma.

In certain embodiments, there is provided a pharmaceutical composition for treatment of RSW or one or more symptoms thereof in an individual, e.g., an individual having an unknown volume status, comprising a salt and water, or comprising an HPR antagonist, wherein: the amount of plasma or serum HPR in a test sample obtained from an individual having RSW, or having one or more symptoms of RSW, relative to the amount of plasma or serum HPR in a normal control describing the amount of plasma or serum HPR in a normal individual is determined; and wherein salt and water, or wherein an HPR antagonist, is administered to the individual where the amount of plasma or serum HPR in the test sample is more than the normal control, thereby treating the individual for RSW or one or more symptoms thereof.

In certain embodiments, there is provided a method for treating an individual for SIADH, or for one or more symptoms thereof comprising: providing, or having provided, a control describing the amount of plasma or serum HPR in a normal individual; assessing, or having assessed, a test sample obtained from an individual for whom SIADH, or one or more symptoms thereof is to be treated, to determine the amount of plasma or serum HPR contained in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is the greater than the amount of plasma or serum HPR in the control, not treating the individual with a vasopressin receptor antagonist, thereby treating the individual for SIADH, or for one or more symptoms thereof.

In certain embodiments, there is provided a method of treating an individual suffering from SIADH, the method comprising the steps of: providing, or having provided, a control describing the amount of plasma or serum HPR in a normal individual; assessing, or having assessed, a test sample obtained from an individual for suffering from SIADH, to determine the amount of plasma or serum HPR contained in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is greater than the amount of plasma or serum HPR in the control, not treating the individual with a vasopressin receptor antagonist, wherein a risk of prolonged SIADH in an individual having an amount of plasma or serum HPR that is the same as or less than the control is lower following vasopressin receptor antagonist administration, than is the risk in an individual having a greater amount of plasma or serum HPR than the control following vasopressin receptor antagonist administration; thereby treating an individual suffering from SIADH.

In certain embodiments, there is provided a method for treating an individual having symptoms of SIADH to minimize said symptoms, the method comprising the following steps: providing, or having provided, a control describing the amount of plasma or serum HPR in a normal individual; assessing, or having assessed, a test sample obtained from an individual for whom one or more symptoms of SIADH are to be minimized, to determine the amount of plasma or serum HPR contained in the test sample; comparing, or having compared, the amount of plasma or serum HPR in the test sample with the control to determine whether the individual has an amount of plasma or serum HPR that is the same as or less than the control; and if the amount of plasma or serum HPR in the test sample is the same as or less than the amount of plasma or serum HPR in the control, treating the individual with a vasopressin receptor antagonist and; if the amount of plasma or serum HPR in the test sample is greater than the amount of plasma or serum HPR in the control, not treating the individual with a vasopressin antagonist, wherein a risk of lesser minimization of symptoms of SIADH in an individual having an amount of plasma or serum HPR that is the same as or less than the control is lower following vasopressin receptor antagonist administration, than is the risk of lesser minimization of symptoms of SIADH in an individual having a greater amount of plasma or serum HPR than the control following vasopressin receptor antagonist administration; thereby treating an individual having symptoms of SIADH to minimize said symptoms.

In certain of the above described embodiments, for example where the amount of plasma or serum HPR in the test sample is greater than the amount of plasma or serum HPR in the control, or the amount of plasma or serum HPR related peptide in the test sample is greater than the HPR related peptide in the control, or the amount HPR signal peptide deletion variant is greater than the amount of plasma or serum HPR signal peptide deletion variant in the control, the individual is more likely to have hypovolemic hyponatremia and therefore is not treated with a vasopressin antagonist. Instead the individual can be treated with other therapies available for hypovolemic hyponatremia including isotonic saline administration.

In certain embodiments, an individual treated according to the above described SIADH-related embodiments will have an unknown volume status. In certain embodiments, the individual is non-oedematous.

In certain embodiments, there is provided a method for treating an individual for SIADH, or for one or more symptoms thereof, where the individual has an unknown volume status, comprising: providing or having provided a control describing the amount of plasma or serum HPR in a normal individual; assessing or having assessed a test sample obtained from an individual for whom SIADH or one more symptoms thereof is to be treated, to determine the amount of plasma or serum HPR contained in the test sample; comparing or having compared the amount of plasma or serum HPR in the test sample with the control; treating the individual with a vasopressin receptor antagonist where the amount of plasma or serum HPR in the test sample is the same as or less than the amount of plasma or serum HPR in the control; thereby treating the individual for SIADH or one or more symptoms thereof.

In certain embodiments, there is provided a composition comprising a vasopressin receptor antagonist, for use in minimizing SIADH or one or more symptoms of SIADH in an individual, e.g., where the individual has an unknown volume status wherein: the amount of plasma or serum HPR in a test sample obtained from an individual having SIADH or one or more symptoms of SIADH relative to the amount of plasma or serum HPR in a normal control describing the amount of plasma or serum HPR in a normal individual is determined; and wherein vasopressin receptor antagonist is utilized to treat the individual where the amount of plasma or serum HPR in the test sample is the same as or less than the normal control, thereby minimizing SIADH, or one or more symptoms of SIADH in an individual. In certain embodiments, the test sample is obtained from plasma or urine, although as described herein the test sample may be obtained from other body fluids or tissues. The control may describe an amount of plasma or serum HPR of about 40 μg/ml.

In certain embodiments, there is provided a pharmaceutical composition for treatment of SIADH or one or more symptoms thereof in an individual, e.g., an individual having an unknown volume status, comprising a vasopressin receptor antagonist, wherein: the amount of plasma or serum HPR in a test sample obtained from an individual having SIADH, or having one or more symptoms of SIADH, relative to the amount of plasma or serum HPR in a normal control describing the amount of plasma or serum HPR in a normal individual is determined; and wherein vasopressin receptor antagonist, is administered to the individual where the amount of plasma or serum HPR in the test sample is the same as or less than the normal control, thereby treating the individual for SIADH or one or more symptoms thereof.

In certain embodiments, the individual that is the subject of the treatment methods is non-oedematous.

In certain embodiments, the individual that is the subject of the treatment methods may be hyponatremic or normonatremic. In certain embodiments, a subject that is normonatremic may be normonatremic as a result of early intervention to increase serum sodium.

In certain embodiments, the subject of the treatment methods is euvolemic, although this volume status is, in certain embodiments, not known at the time of the application of the treatment methods herein.

In certain embodiments, the individual that is the subject of the treatment method can have increased fractional excretion of urate (FEU). In certain embodiments, the FEU is >11%.

In certain embodiments, the individual that is the subject of the treatment method can be hypouricemic. In certain embodiments, the serum concentration of uric acid of the individual is <4 mg/dL.

In certain embodiments, the individual that is the subject of the treatment method can have concentrated urine. In certain embodiments, the concentration of urine is <300 mosm/kg or >Posm.

In certain embodiments, the individual that is the subject of the treatment method can have high urine sodium concentration. In certain embodiments, the sodium concentration of >30 mEq/L. In certain embodiments, it can be lower than 30 mEq/L.

In certain embodiments, the individual that is the subject of the treatment method is non-oedematous, hyponatremic, hypouricemic, has a high urine sodium concentration, a high fractional excretion of urate and concentrated urine.

In certain embodiments, the individual that is the subject of treatment is not a person who has oedema or who has hypervolemia arising from cardiac or other organ failure or dysfunction.

In certain embodiments, the symptoms of hyponatremia can be one or more of unsteady gait, weakness, nausea, malaise, lethargy, confusion, decreased mental capacity, decreased level of consciousness, headache, seizures and coma.

The symptoms of hyponatremia can be associated with acute or chronic hyponatremia. Symptoms of acute hyponatremia may present generally no longer than a day or two days. The symptoms of RSW can be associated with acute or chronic RSW.

Symptoms of chronic hyponatremia can be present for longer than 1 month, e.g., 1 to 6 months or longer including years depending on the comorbid condition.

In certain embodiments, the individual has symptoms of acute hyponatremia including mental status changes, agitation, seizure or neurogenic pulmonary oedema. In certain embodiments, the individual can have a further acute disease or disorder or an adverse drug reaction. Where the individual has hyponatremic symptoms comorbid with an acute condition, the individual may be normonatremic or hyponatremic. Again, in certain embodiments, the individual is more likely normonatremic where he/she has been prior treated to elevate serum sodium and will eventually return to low serum sodium unless treated by the methods described herein. In certain embodiments, if the individual has symptoms of acute hyponatremic, the individual can have hypertrophic cells. In certain embodiments, the symptoms of hyponatremia are associated with chronic hyponatremia. In certain embodiments, the individual can have a further chronic disease or disorder. In certain embodiments, the individual has chronic asymptomatic hyponatremia.

In certain embodiments, the individual has symptoms of acute RSW. In certain embodiments, the individual can have a further acute disease or disorder or an adverse drug reaction. Where the individual has RSW symptoms comorbid with an acute condition, the individual may be normonatremic or hyponatremic. In certain embodiments, the individual has symptoms of chronic RSW. In certain embodiments, the individual can have a further chronic disease or disorder, e.g., Alzheimer's or other neurological disorder. Where the individual has chronic RSW symptoms comorbid with a chronic condition, the individual may be normonatremic or hyponatremic.

In certain embodiments, the outcome of the therapeutic methods described herein is the minimization, and sometimes, ablation of one or more symptoms of hyponatremia.

It will be understood that where the same symptoms arise from comorbid indications that are not treated by salt and water, or not treated by HPR antagonist therapy, the methods described herein can be more applicable to preventing the worsening or development of hyponatremic symptoms, rather than minimization or ablation of symptoms.

In certain embodiments, the individual that is the subject of the treatment method may be assessed for one or more of the following parameters: serum sodium concentration, urine sodium concentration, serum uric acid concentration, during, after or prior to salt and water, or HPR antagonist therapy, or in the circumstances, water treating. These parameters can be assessed by standard techniques during, prior to or after the treatment methods described herein.

In certain embodiments, the individual that is the subject of treatment will not be assessed to determine the volume status of the individual during or after the methods described herein. In certain embodiments, the volume status of the individual will be unknown.

Salt and water treatment therapy for RSW syndrome is generally well described in the art. This therapy is generally applicable in the methods of the invention described herein. In certain embodiments, where water treatment is implemented, salt in the form of sodium salt may also be administered. In the case of water treatment, salt may be provided together with, or separate to water treatment.

The outcome of the therapeutic methods described herein is generally the minimization, and sometimes, ablation of one or more symptoms of hyponatremia and RSW. It will be understood that where the same symptoms arise from comorbid indications that are not treated by salt and water treatment, the methods described herein can be more applicable to preventing the worsening or development of hyponatremic symptoms, rather than minimization or ablation of symptoms.

In certain embodiments, the individual the subject of the treatment methods disclosed herein can be assessed for one or more of the following parameters: serum sodium concentration, urine sodium concentration, serum uric acid concentration, during after or prior to water-restricting or water treating. These parameters can be assessed by standard techniques during or prior to the treatment methods described herein.

In certain embodiments, the individual that is the subject of treatment will not be assessed to determine the volume status of the individual. In certain embodiments, the volume status of the individual will be unknown.

In certain embodiments, there is provided a method for treating an individual having one or more symptoms of hyponatremia including administering an HPR antagonist to the individual, thereby treating the individual for one or more symptoms of hyponatremia. In certain embodiments, the individual has RSW syndrome. In certain embodiments, the HPR antagonist is an anti-HPR antibody, examples of which are described in the following sub-heading. In certain embodiments, the HPR antagonist is a peptide having an HPR related sequence, enabling competitive inhibition of binding of HPR to proximal convoluted tubule cells. In certain embodiments, there is provided a pharmaceutical composition including an HPR antagonist and a pharmaceutically acceptable diluent, excipient or carrier.

In certain embodiments, the HPR antagonist is provided in a pharmaceutically acceptable amount. A pharmaceutically acceptable amount of an HPR antagonist may be an amount enabling the reduction of serum HPR concentration to about 40 μg/mL or less. In certain embodiments, the pharmaceutical composition is provided in the form of a formulation adapted for IV administration although other forms specific for other administration routes such as oral administration are contemplated.

In certain embodiments, an HPR antagonist for treatment of salt wasting syndrome is provided in the form of a recombinant or synthetic peptide.

In one embodiment, the peptide antagonist may be provided in the form of an a subunit of HPR. An a subunit of HPR has a sequence shown in SEQ ID No: 2.

In certain embodiments, the peptide antagonist has a sequence that is at least 91%, at least 95%, at least 96%, or 97% or 98%, or 99% homologous to the sequence of SEQ ID No:2, or a sequence that is identical to the sequence of SEQ ID No:2. A sequence that is at least 91%, at least 95%, at least 96%, or 97% or 98%, or 99% homologous to the sequence of SEQ ID No:2 is referred to as a “variant of SEQ ID No: 2”.

Percentage homology is generally assessed with reference to a comparison window of about 6 to 12 contiguous residues with a reference sequence (which may be a sequence of SEQ ID No: 1, 2 or 3). The comparison window may comprise additions or deletions (i.e., gaps) of about 20% or less as compared to the reference sequence for optimal alignment of the respective sequences. Optimal alignment of sequences for aligning a comparison window may be conducted by computerized implementations of algorithms or by inspection and the best alignment (i.e. resulting in the highest percentage homology over the comparison window) generated by any of the various methods selected. Reference also may be made to the BLAST family of programs as for example disclosed by Altschul et al, 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-2015).

In certain embodiments, a variant of a reference sequence (a reference sequence being SEQ ID No: 1 (or 2 or 3 described below)) will differ from the reference sequence by no more than about 1 to 5 amino acid residues, typically no more than about 1 to 2 amino acid residues, or 1 amino acid residue.

In certain embodiments, the peptide antagonist may include or consist of the sequence of SEQ ID No: 2, or may include or consist of the sequence of a variant of SEQ ID No: 2. In certain embodiments, the peptide antagonist may be provided in the form of a fragment of a sequence of SEQ ID No: 2, or a fragment of a variant of SEQ ID No:2.

In one embodiment, the peptide antagonist may be provided in the form of a β subunit of HPR. A p subunit of HPR has a sequence shown in SEQ ID No: 3.

In certain embodiments, the peptide antagonist has a sequence that is at least 91%, at least 95%, at least 96%, or 97% or 98%, or 99% homologous to the sequence of SEQ ID No: 3, or a sequence that is identical to the sequence of SEQ ID No: 3. A sequence that is at least 91%, at least 95%, at least 96%, or 97% or 98%, or 99% homologous to the sequence of SEQ ID No: 3 is referred to as a “variant of SEQ ID No: 3”. Percentage homology can be determined as discussed above.

In certain embodiments, the peptide antagonist may include or consist of the sequence of SEQ ID No: 3, or may include or consist of the sequence of a variant of SEQ ID No: 3.

In certain embodiments, the peptide antagonist may be provided in the form of a fragment of a sequence of SEQ ID No: 3, or a fragment of a variant of SEQ ID No: 3.

In certain embodiments, the peptide antagonist may be provided in the form of a peptide having a sequence shown in SEQ ID No: 1.

In certain embodiments, the peptide antagonist has a sequence that is at least 91%, at least 95%, at least 96%, or 97% or 98%, or 99% homologous to the sequence of SEQ ID No: 1, or a sequence that is identical to the sequence of SEQ ID No: 1. A sequence that is at least 91%, at least 95%, at least 96%, or 97% or 98%, or 99% homologous to the sequence of SEQ ID No: 1 is referred to as a “variant of SEQ ID No: 1”. Percentage homology can be determined as discussed above.

In certain embodiments, the peptide antagonist may include or consist of the sequence of SEQ ID No: 1, or may include or consist of the sequence of a variant of SEQ ID No: 1.

In certain embodiments, the peptide antagonist may be provided in the form of a fragment of a sequence of SEQ ID No: 1, or a fragment of a variant of SEQ ID No: 1.

In certain embodiments, the peptide antagonist is a fragment of a sequence of SEQ ID No: 1, 2 or 3, or a fragment of a variant of SEQ ID No: 1, 2 or 3, wherein the peptide is a proximal convoluted tubule cell receptor antagonist i.e. the peptide competitively inhibits the binding of HPR to a receptor on a proximal cell that, in the absence of the peptide, is bound by HPR. A fragment may generally be from about a 5-mer to a 100-mer. Assays for assessing competitive inhibition of peptide binding to cell surface receptors, such as radio-isotope assays known in the art could be utilized to identify peptides that competitively inhibit the binding of HPR to a receptor on a proximal cell.

In certain embodiments, an HPR antagonist in the form of a peptide antagonist comprises an HPR signal peptide. In certain embodiments, an HPR antagonist is anti-hyponatremic. In certain embodiments, an HPR antagonist is not a diuretic.

In certain embodiments, a peptide that is an HPR-related peptide antagonist includes an HPR specific amino acid sequence, more particularly, a sequence of amino acids that is found in HPR but not found in haptoglobin (HP). These sequences can be determined from alignment of the HPR sequence (SEQ ID No: 1) with the sequence of HP (SEQ ID No: 4) according to standard techniques.

In certain embodiments, the peptide antagonist is provided in the form of a molecule that binds to HPR, examples including haemoglobin, haemoglobin binding peptide and paraoxonase-arylesterase.

Where the HPR antagonist is a peptide, the amount can be 1 to 1000 μg/mL, 10 to 500 μg/ml, 10 to 100 μg/ml, about 10 to 50 or 25 to 50 μg/ml.

In certain embodiments, a peptide antagonist may be cyclized or otherwise modified to improve stability or half-life in vivo.

In certain embodiments, an HPR antagonist for treatment of salt wasting syndrome is provided in the form of an anti-HPR antibody. In certain embodiments, HPR antibodies can be provided in the form of monoclonal antiserum, or polyclonal antiserum. In certain embodiments, the antibody is a monoclonal antibody. In certain embodiments, the antibody may be provided in the form of whole antibody, or antibody fragment that has HPR binding variable domains. Examples of fragments include dAbs, fAbs, single chain antibodies and variable regions. In certain embodiments, an anti HPR antibody is generally selective for binding to HPR, meaning that the antibody does not bind to haptoglobin.

In certain embodiments, an anti HPR antibody can bind only to the HPR a subunit, only to the HPR subunit, or to both a and p subunits. An antibody that binds to both a and p subunits can be bi-specific, or can include a variable domain that binds to an epitope arising from contributions from the a and p subunits.

In certain embodiments, the antibody binds to epitopes presented on the heterodimer formed from disulfide bonding of the a subunit with the p subunit. In certain embodiments, the binding of the antibody to serum HPR heterodimers may lead to the clearance or removal of HPR from serum.

In certain embodiments, the antibody binds to HPR that is contained within a apolipoprotein L-1 (apo-L-1)-containing high density lipoprotein (HDL) particle.

In certain embodiments, the amount of plasma or serum HPR antagonist in the form of anti-HPR antibodies may be 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg kg, etc.), of the host body weight. For example, dosages can be 1 mg/kg body weight or 10 mg/kg body weight or within the range of 1-60 mg/kg, or at least 1 mg/kg. Doses intermediate in the above ranges are also intended to be within the scope of the invention.

HPR antibodies for use as HPR antagonists can be formed by immunization with an HPR antagonist peptide described above, or by immunization with serum HPR. HPR antibodies are commercially available, e.g., as described in the Examples below.

In certain embodiments, there is provided a method for treatment of an individual having renal salt wasting or symptom thereof including administering an HPR antagonist in the form of a peptide antagonist, anti-HPR antibody or HPR binding molecule, as described herein, to an individual requiring said treatment, thereby treating the individual for renal salt wasting or symptom thereof. In certain embodiments, the individual can have a co-morbid condition including bone fracture or brain injury or neurological disease.

In certain embodiments, there is provided an HPR antagonist in the form of a peptide antagonist, anti-HPR antibody or HPR binding molecule, as described herein, for use in the treatment of an individual having renal salt wasting or symptom thereof. The individual can have a co-morbid condition including bone fracture or brain injury or neurological disease.

In certain embodiments, there is provided a use of an HPR antagonist in the form of a peptide antagonist, anti-HPR antibody or HPR binding molecule, as described herein, in the manufacture of a medicament for treatment of an individual having renal salt wasting or symptom thereof. The individual can have a co-morbid condition including bone fracture or brain injury or neurological disease.

### 2.2 Methods of Diagnosis Based on Amount of HPR

In certain embodiments, there is provided a method for determining whether an individual has RSW or SIADH comprising: determining the amount of plasma or serum HPR in a test sample obtained from an individual having RSW or SIADH, or having one or more symptoms of RSW or SIADH, relative to the amount of plasma or serum HPR in a normal control describing the amount of plasma or serum HPR in a normal individual, wherein an amount of plasma or serum HPR in the test sample that is more than the normal control determines that the individual has RSW, while if the sample has the same or less that n the normal control, the individual has SIADH. In certain embodiments, the test sample is obtained from plasma or urine, although as described herein the test sample may be obtained from other body fluids or tissues. In certain embodiments, the control contains an amount of HPR of about 40 μg/ml in plasma or serum.

In certain embodiments, there is provided an HPR selective agent for use in determining whether an individual has RSW or SIADH comprising: utilizing an HPR selective agent to determine the amount of plasma or serum HPR in a test sample obtained from an individual having RSW or SIADH, or having one or more symptoms of RSW or SIADH relative to the amount of plasma or serum HPR in a normal control describing the amount of plasma or serum HPR in normal individual; wherein an amount of plasma or serum HPR in the test sample that is more than the normal control determines that the individual has RSW, while an amount of plasma or serum HPR that is the same or less than the normal control determines the individual has SIADH.

### 2.3 Kits Based on Amount of HPR

In certain embodiments, there is provided a kit for determining whether an individual has RSW syndrome or for use in providing treatment for an individual having hyponatremia or one or more symptoms thereof comprising: a reagent, e.g., an HPR selective agent, for determining the amount of plasma or serum HPR in a test sample obtained from an individual for whom the presence of RSW syndrome is to be determined, or for whom treatment is to be provided; written instructions for use in a method described above.

In certain embodiments, the kit contains data enabling the establishment of a control describing the amount of plasma or serum HPR in a normal individual and/or a control describing the amount of plasma or serum HPR in an individual having RSW.

For example, but not by way of limitation, in the methods described herein, various controls may be implemented. In certain embodiments, a normal control is a control describing the amount of plasma or serum HPR in a normal individual.

In certain embodiments, a normal individual is an individual who does not have significant symptoms of hyponatremia. In certain embodiments, a normal individual does not have one or more of increased FEUA, and hypouricemia and hyponatremia. In certain embodiments, a normal individual does not have increased urine salt concentration, decreased serum salt concentration or form concentrated urine. In certain embodiments, a normal individual does not have abnormal vascular volume. In certain embodiments, the methods for measuring urine and serum sodium concentrations are accomplished by standard automated methods, concentration of urine Fiske Osmometer or other methods of measuring volume of different spaces of the body, such as vascular volume by 51 chromium red blood cells or radioiodinated serum albumin, total body by deuterium and extracellular volume by sulfate or bromide.

In certain embodiments, the control describes the amount or concentration of HPR in plasma or serum of a normal individual. In certain embodiments, the concentration of HPR in plasma or serum of a normal individual is about 35 to about 80 μg/ml, about 35 to about 70 μg/ml, about 35 to about 60 μg/ml, about 35 to about 50 μg/ml, about 35 to about 45 μg/ml, or about 40 μg/ml.

In certain embodiments, a normal control can be utilized where the method is performed on the basis of quantification of a HRP related peptide or a HRP signal peptide deletion variant. In certain embodiments, the control can be derived from a single normal individual. However, in certain embodiments, the control is derived from a cohort of normal individuals.

As described herein, a purpose of the control is to provide a reference point against which a determination regarding implementation of subsequent therapy can be made. The determination can be made on the basis of the comparison between test sample and control. In certain embodiments, the comparison can be as between the serum amount or concentration of HPR in the test sample and the control. It will be understood that, in certain embodiments, the control may be provided in the form of data that has been derived prior to assessment of the subject for treatment.

In certain embodiments, the method can include the further step of comparing the amount of plasma or serum HPR in the test sample with a further control in the form of an RSW control. For example, but not by way of limitation, the RSW control describes the amount of plasma or serum HPR in an individual who has been identified as having RSW. For example, the individual may have been identified as having RSW on the basis of assessment of serum sodium concentration and FEUA.

The amount of plasma or serum HPR in the RSW control can be expressed as the serum concentration of HPR in the individual who has been identified as having RSW. In certain embodiments, the amount is more than 40 μg/ml, generally less than about 100 μg/ml. In certain embodiments, an RSW control may be derived from a single RSW individual. However, in some embodiments the control is derived from a cohort of RSW individuals.

As described herein, in certain embodiments, a purpose of the control can be to provide a reference point against which a determination regarding implementation of appropriate treatment can be made. In certain embodiments, the determination may be made on the basis of the comparison between test sample and RSW control. For example, the comparison can be as between the serum amount or concentration of HPR in the test sample and the RSW control. It will be understood that, in certain embodiments, the RSW control can be provided in the form of data that has been derived prior to assessment of the subject for treatment.

In certain embodiments, there is provided a method for treating an individual for hyponatremia or one or more symptoms thereof comprising: assessing or having assessed a test sample obtained from an individual in whom at least one or more symptoms of hyponatremia are to be minimized to determine the amount of plasma or serum HPR in the test sample; comparing or having compared the amount of plasma or serum HPR in the test sample with an RSW control, the RSW control describing the amount of plasma or serum HPR in an individual who has been identified as having RSW; salt and water-treating the individual, or treating the individual with an HPR antagonist, where the amount of plasma or serum HPR in the test sample is the same or more than the amount of plasma or serum HPR in the RSW control; thereby treating the individual to at least minimize one or more symptoms of hyponatremia.

In certain embodiments, there is provided a method for treating an individual to at least minimize one or more symptoms of hyponatremia in the individual comprising: assessing or having assessed a test sample obtained from an individual in whom at least one or more symptoms of hyponatremia are to be minimized to determine the amount of plasma or serum HPR in the test sample; comparing or having compared the amount of plasma or serum HPR in the test sample with a normal control, the normal control describing the amount of plasma or serum HPR in a normal individual; comparing or having compared the amount of plasma or serum HPR in the test sample with an RSW control, the RSW control describing the amount of plasma or serum HPR in an individual who has been identified as having RSW; salt and water-treating the individual, or treating the individual with an HPR antagonist, where the amount of plasma or serum HPR in the test sample is greater than the amount of plasma or serum HPR in the normal control, and the same as or greater than the amount of plasma or serum HPR in the RSW control; thereby treating the individual to at least minimize one or more symptoms of hyponatremia.

According to certain embodiments, the individual the subject of the method is to be salt water-treated, or treated with an HPR antagonist where: the amount of plasma or serum HPR in the test sample is greater than the amount of plasma or serum HPR in the normal control; and/or the amount of plasma or serum HPR in the test sample is the same as, or more than the amount of plasma or serum HPR in the RSW control.

In certain embodiments, there is provided a kit for determining whether an individual has SIADH syndrome, or other euvolemic hyponatremic disorder, or for use in providing treatment for an individual having hyponatremia or one or more symptoms thereof comprising: a reagent, e.g., an HPR selective agent, for determining the amount of plasma or serum HPR in a test sample obtained from an individual for whom the presence of SIADH syndrome, or other euvolemic hyponatremic disorder is to be determined, or for whom treatment is to be provided; written instructions for use in a method described above.

In certain embodiments, the kit contains data enabling the establishment of a control describing the amount of plasma or serum HPR in a normal individual and/or a control describing the amount of plasma or serum HPR in an individual having SIADH or other euvolemic hyponatremic disorder.

In certain embodiments of methods described herein, various controls may be implemented. For example, a normal control can be a control describing the amount of plasma or serum HPR in a normal individual. In certain embodiments, a normal individual is an individual who does not have significant symptoms of hyponatremia. In certain embodiments, a normal individual does not have one or more of increased FEUA, and hypouricemia and hyponatremia. In certain embodiments, a normal individual does not have increased urine salt concentration, decreased serum salt concentration or form concentrated urine. In certain embodiments, a normal individual does not have abnormal vascular volume.

In certain embodiments, methods for measuring urine and serum sodium concentrations are generally by standard automated methods, concentration of urine Fiske Osmometer or other methods of measuring volume of different spaces of the body, such as vascular volume by 51 chromium red blood cells or radioiodinated serum albumin, total body by deuterium and extracellular volume by sulfate or bromide.

In certain embodiments, the control describes the amount or concentration of HPR in serum of a normal individual. In certain embodiments, the concentration of HPR in plasma of a normal individual is about 35 to about 80 μg/ml, about 35 to about 70 μg/ml, about 35 to about 60 μg/ml, about 35 to about 50 μg/ml, about 35 to about 45 μg/ml, or about 40 μg/ml.

In certain embodiments, a normal control can be utilized where the method is performed on the basis of quantification of a HRP related peptide or a HRP signal peptide deletion variant. In certain embodiments, the control is derived from a single normal individual. However, in certain embodiments, the control is derived from a cohort of normal individuals.

In certain embodiments, as described herein, a purpose of the control is to provide a reference point against which a determination regarding implementation of subsequent vasopressin receptor antagonist therapy can be made. In certain embodiments, the determination is made on the basis of the comparison between test sample and control. The comparison is can be between the serum amount or concentration of HPR in the test sample and the control.

In certain embodiments, the control can be provided in the form of data that has been derived prior to assessment of the subject for treatment.

In certain embodiments, the method may include the step of comparing the amount of plasma or serum HPR in the test sample with an SIADH control. In certain embodiments, the SIADH control describes the amount of plasma or serum HPR in an individual who has been identified as having SIADH. In certain embodiments, the individual may have been identified as having SIADH on the basis of assessment of serum sodium concentration and FEUA.

In certain embodiments, the amount of plasma or serum HPR in the SIADH control is generally expressed as the serum concentration of HPR in the individual who has been identified as having SIADH. In certain embodiments, the amount is less than about 40 μg/ml. In certain embodiments, a SIADH control may be derived from a single SIADH individual. However, in some embodiments, the control is derived from a cohort of SIADH individuals.

In certain embodiments, as described herein, a purpose of the control is to provide a reference point against which a determination regarding implementation of appropriate treatment can be made. In certain embodiments, the determination is made on the basis of the comparison between test sample and SIADH control. In certain embodiments, the comparison is between the serum amount or concentration of HPR in the test sample and the SIADH control. It will be understood that, in certain embodiments, the SIADH control may be provided in the form of data that has been derived prior to assessment of the subject for treatment.

In certain embodiments, the reagent for determining the amount of plasma or serum HPR is an antibody, e.g., a monoclonal antibody that binds to HPR or a fragment thereof.

In certain of the methods and kits described herein, antibodies against HPR can be labelled with a detectable moiety. The detectable moiety can be any one which is capable of producing, either directly or indirectly, a detectable signal. For example, the detectable moiety can be a radioisotope, such as 3H, 14C, 32P, 35S, or 125; a fluorescent or chemiluminescent compound (Melegos et al., Clin. Chem. 42:12 (1996)), such as fluorescein isothiocyanate, rhodamine, or luciferin; radioactive isotopic labels, such as, e.g., 1251, 32P, 14C, or 3H; or an enzyme, such as alkaline phosphatase, beta-galactosidase, or horseradish peroxidase.

Any method known in the art for separately conjugating the antibody to the detectable moiety can be employed, including those methods described by Hunter et al., Nature, 144: 945 (1962); David et al., Biochemistry, 13: 1014 (1974); Pain et al., J. Immunol. Meth., 40: 219 (1981); and Nygren, J. Histochem. and Cytochem., 30: 407 (1982).

The antibodies used in the methods and kits can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labelled standard (which can be HPR or an immunologically reactive portion thereof) to compete with the test sample for binding with a limited amount of antibody. In certain embodiments, the amount of plasma or serum HPR in the test sample is inversely proportional to the amount of standard that becomes bound to the antibodies. To facilitate determining the amount of standard that becomes bound, the antibodies generally are insolubilized before or after the competition, so that the standard and HPR from the tested sample that are bound to the antibodies may conveniently be separated from the unbound material.

In certain embodiments, sandwich assays involve the use of two antibodies, each capable of binding to a different immunogenic portion, or epitope, of HPR to be detected. In certain sandwich assays, HPR is bound by a first antibody which is immobilized on a solid support, and thereafter a second antibody binds to the protein, thus forming an insoluble three-part complex. David and Greene, U.S. Pat. No. 4,376,110. The second antibody can itself be labelled with a detectable moiety (direct sandwich assays) or can be measured using an anti-immunoglobulin antibody that is labelled with a detectable moiety (indirect sandwich assay). For example, one type of sandwich assay is an ELISA assay (Enzyme Linked immunoabsorbent assay), in which case the detectable moiety is an enzyme (e.g., horseradish peroxidase).

Anti-HPR antibodies are useful in diagnostic assays for HPR, e.g., its production in specific cells or tissues, or its presence in urine or serum. In certain embodiments, the antibodies are labelled and/or are immobilized on an insoluble matrix. In certain embodiments, an antibody that binds to HPR is immobilized on an insoluble matrix, the test sample is contacted with the immobilized antibody composition to adsorb all HPR, and then the immobilized HPR are contacted with antibodies that recognize different antigenic sites on HPR, these antibodies being identifiable by a unique label such as discrete fluorophores or the like. By determining the presence and/or amount of the unique label, the amount of plasma or serum HPR can be determined.

In certain embodiments, competitive assays rely on the ability of a tracer (i.e. labelled) analogue to compete with the test sample HPR for a limited number of binding sites on a common binding partner. The binding partner can be insolubilized before or after the competition and then the tracer and HPR bound to the binding partner are separated from the unbound tracer and HPR. This separation is accomplished by decanting (where the binding partner was pre-insolubilized) or by centrifuging (where the binding partner was precipitated after the competitive reaction). In certain embodiments, the amount of test sample HPR peptide is inversely proportional to the amount of bound tracer as measured by the amount of marker substance. Dose-response curves with known amounts of HPR are prepared and compared with the test results to quantitatively determine the amount of plasma or serum HPR present in the test sample. These assays are called ELISA systems when enzymes are used as the detectable markers.

Sandwich assays particularly are useful for the determination of HPR. In sequential sandwich assays an immobilized binding partner is used to adsorb test sample HPR, the test sample is removed as by washing, the bound HPR is used to adsorb labelled binding partner, and bound material is then separated from residual tracer. The amount of bound tracer is directly proportional to test sample HPR. In “simultaneous” sandwich assays the test sample is not separated before adding the labelled binding partner. A sequential sandwich assay using an anti-HPR monoclonal antibody as one antibody and a polyclonal anti-HPR as the other is useful in testing samples for HPR presence.

Other reagents for selectively detecting and quantifying the amount of plasma or serum HPR in a test sample or control as described herein could be utilized in the methods and kits described herein.

In certain embodiments, the samples to be tested are bodily fluids such as blood, serum, plasma, urine, tears, saliva and the like.

In certain embodiments, the sample from the individual will require processing prior to detection of the levels of HPR. For example, the sample may be centrifuged or diluted to a particular concentration or adjusted to a particular pH prior to testing. Conversely, it may be desirable to concentrate a sample that is too dilute, prior to testing.

It will be understood that the methods of the invention may be based on detection of the whole of the HPR protein, or on protein fragments thereof that have amino acid sequences that distinguish those fragments as arising from HPR instead of some other polypeptide, such as Haptoglobin. As discussed herein, exemplary peptides that are unique to HPR and not found in haptoglobin can be determined by alignment of HPR amino acid sequence with the haptoglobin amino acid sequence, not including alignment of leader sequences.

In certain embodiments, the sample may be processed by protease digestion, for example by trypsin digestion, and proteolytic fragments determined or measured to determine the presence or quantify the amount of HRP, HRP related fragment or HRP signal peptide deletion variant.

In certain embodiments, HRP may be detected in the form of a dimer or heterodimer, or complex with another protein, carbohydrate of lipid. For example, the HPR may be detected in the form of a high-density lipoprotein bound molecule such as a trypanosome complex.

In certain embodiments, the presence or absence or amount of plasma or serum HPR or fragment thereof or HPR signal peptide deletion variant can be detected on the basis of expression of RNA or other nucleic acid utilizing a nucleic acid probe.

In certain embodiments, a selective HPR nucleic acid probe is utilized that enables the selective detection of nucleic acids encoding HPR, but not the detection of nucleic acid sequences encoding haptoglobin.

In certain embodiments, a selective HPR signal peptide deletion variant nucleic acid probe is utilized that enables the selective detection of nucleic acids encoding an HPR signal peptide deletion variant, but not the detection of nucleic acid sequences encoding haptoglobin or HPR.

In certain embodiments, a nucleic acid probe is utilized to detect gene expression of HPR or a signal peptide deletion variant in a tissue biopsy, e.g., in liver biopsy.

In certain embodiments, a nucleic acid probe is utilized to detect HPR or signal peptide deletion variant in renal tissue, or in urine, e.g., in urine microvesicles.

The inventors have found that individuals having higher than normal amounts of HPR or presence of HPR signal peptide deletion variants in serum or plasma tend also to have higher amounts of haptoglobin forms HP1-1, HP1-2 or HP2-2. While the examples herein demonstrate that haptoglobin does not induce RSW symptoms in a rat model, the inventors note that haptoglobin and HPR genes are tightly linked and may be under influence of similar transcriptional or other gene expression controls. Thus, in certain embodiments, the present disclosure provides methods for determining whether an individual is at risk of hyponatremia or RSW including: determining the amount of haptoglobin in a test sample obtained from an individual in whom risk of hyponatremia or RSW is to be determined relative to the amount of haptoglobin in a normal control describing the amount of haptoglobin in a normal individual; determining a high likelihood of the individual developing RSW where the individual has an amount of haptoglobin relative to the control that is more than the control; determining a low likelihood of the individual developing RSW where the individual has an amount of haptoglobin relative to the control that is the same as or less than the control, thereby determining the likelihood of the individual being at risk of hyponatremia or RSW.

In certain embodiments, the individual has a higher risk for developing RSW where the blood concentration of haptoglobin is >200 to 300 mg/dL. In certain embodiments, the methods disclosed herein can further include assessing the amount of plasma or serum HPR, or for the presence of HPR signal peptide deletion variant in a test sample from the individual.

In certain embodiments, a normal control describing the amount of haptoglobin in a normal individual generally defines an amount of HP of about 126 mg/dl.

### 3. Diuretic Compositions and Methods

A diuretic is a substance that causes increased production of urine. Several categories are recognized on basis of site of action, mode of action, and chemical structure. Diuretics may increase urine flow rate. Urinary flow rate is the quantity of urine excreted in a specified period of time (per second or per minute). Diuretics may also increase the fractional excretion of sodium (FENa). FENa is the percentage of the sodium filtered by the kidney which is excreted in the urine.

In medicine, diuretics are used to minimize edema, particularly in those conditions where edema contributes to increased morbidity, including for example congestive heart failure, chronic kidney disease, nephrotic syndrome and liver cirrhosis. Some individuals develop diuretic resistance. Diuretic resistance can arise from compensatory increases in sodium reabsorption in nephron sites that are not blocked by a diuretic. Oedemic individuals with congestive heart failure have been observed to develop resistance to loop diuretics such as bumetanide, ethacrynic acid, furosemide and torsemide.

Diuretics may selectively interact at various nephron regions: potassium sparing diuretics tend to act at the collecting duct and connecting tubule; thiazides act at the distal convoluted tubule; loop diuretics act at the thick ascending limb of the loop of Henle; and carbonic anhydrase inhibitors and osmotic diuretics act at the proximal convoluted tubule. These discrete sites of action provide opportunity to adjust diuretic therapy in refractory or resistant individuals. As outlined herein, the instant disclosure provides new diuretic compositions and method of inducing diuresis making use of the same.

### 3.1 Diuretic Compositions

In certain embodiments, there is provided a composition comprising: HPR, or an HPR signal peptide deletion variant, as an active principle, e.g., as an active principle for inducing diuresis, or for increasing FENa, or for increasing FELi, or for increasing urine flow rate; and a carrier, excipient or solvent. In certain embodiments, the HPR signal peptide deletion variant does not comprise the HPR signal peptide. For example, the variant does not comprise the sequence shown in SEQ ID No: 7. In certain embodiments, an HPR signal peptide deletion variant does not comprise part of the HPR signal peptide. For example, the variant may comprise only some or part of the sequence shown in SEQ ID No: 7.

In certain embodiments, an HPR signal peptide deletion variant does not comprise some or all of the HPR signal peptide and comprises a sequence that is at least 90% identical to the sequence shown in SEQ ID No:1, provided that the sequence at least 90% identical to the sequence shown in SEQ ID No:1 contains a sequence shown in SEQ ID No: 14, 15, 16, 17, 18, 19 or 20.

In certain embodiments, an HPR signal peptide deletion variant comprises part of the mature HPR sequence (i.e., part of the a chain (i.e., as shown in SEQ ID No:2), or part of the p chain (i.e., as shown in SEQ ID No:3)) and does not comprise the sequence shown in SEQ ID No: 7.

In certain embodiments, an HPR signal peptide deletion variant comprises part of the mature HPR sequence (i.e., part of the a chain (i.e., as shown in SEQ ID No:2), or part of the p chain (i.e., as shown in SEQ ID No:3)) and comprises only some or part of the sequence shown in SEQ ID No: 7.

In certain embodiments, an HPR signal peptide deletion variant comprises or consists of a sequence shown in SEQ ID No: 8, 9, 10, 11 or 12.

In certain embodiments, an HPR signal peptide deletion variant has an N-terminal sequence of the sequence shown in SEQ ID No:2.

In certain embodiments, an HPR signal peptide deletion variant has an N-terminal sequence of the sequence shown in SEQ ID No: 8.

In certain embodiments, HPR for use in a composition described above may have an amino acid sequence as shown in SEQ ID No: 1.

In certain embodiments, HPR for use in a composition described above may have an α subunit that is about 13.5 kD and that contains the amino acid sequence shown in SEQ ID No: 2.

In certain embodiments, HPR for use in a composition described above may have a β subunit that is about 36.5 kD and that contains the amino acid sequence shown in SEQ ID No: 3.

In certain embodiments, HPR for use in a composition described above may include 1 to 5 amino acid differences from the HPR sequence of SEQ ID No: 1.

In certain embodiments, a peptide described herein induces diuresis at a proximal tubule, e.g., selectively induces diuresis at a proximal tubule (i.e., does not substantially induce diuresis at a distal tubule, connecting tubule or collecting duct). Diuresis at a proximal tubule may be assessed by measuring FELi according to the methods described herein.

In certain embodiments, the HPR or HPR signal peptide deletion variant is provided in a composition in an amount of about 10 to 200 mg per patient per treatment.

In certain embodiments, the HPR or HPR signal peptide deletion variant is provided in a composition for IV delivery in an amount of about 10 to 200 mg per patient per treatment.

In certain embodiments, the HPR or HPR signal peptide deletion variant may be produced by standard techniques including solid phase synthesis and recombinant expression. In certain embodiments, the HPR may be obtained by purification or fractionation of serum or plasma.

In certain embodiments, the composition may be adapted to enable administration by intra-venous (IV), oral, rectal or other route conventionally used for administration of a pharmaceutical composition. In certain embodiments, the composition is provided in a form enabling IV administration. In certain embodiments, the composition includes a carrier, excipient or solvent for intravenous use. Methods for formulation of IV compositions are well known in the art.

In certain embodiments, the HPR or HPR signal peptide deletion variant will be defined by particular advantages. For example, one particular advantage of HPR or HPR signal peptide deletion variants, e.g., those peptides having a canonical amino acid sequence, is that they have an established plasma half-life and therefore can be reasonably expected to persist in plasma for at least the period of the established plasma half-life. In certain embodiments, HPR canonical sequences have low allo-immunogenicity given that HPR has been found to have a highly conserved amino acid sequence.

### 3.2 Methods of Inducing Diuresis

In certain embodiments, there is provided a method for inducing diuresis in an individual comprising administering haptoglobin related protein (HPR), e.g., an HPR signal peptide deletion variant to an individual in whom diuresis is to be induced, thereby inducing diuresis in the individual. In certain embodiments, the method increases fractional excretion of sodium (FENa) in an individual. In certain embodiments, the method increases urinary flow rate in an individual.

In certain embodiments, the individual that is the subject of the method can have oedema. In certain embodiments, the individual does not have oedema and is hypervolemic.

In certain embodiments, the individual may or may not have clinical condition. In certain embodiments, the individual has a chronic condition associated with oedema. In certain embodiments, the individual has a condition selected from the group consisting of nephrotic syndrome, chronic kidney disease, congestive heart failure and liver cirrhosis.

In certain embodiments, the individual can be euvolemic or hypovolemic and hyponatremic. For example, but not by way of limitation, the individual can have syndrome of inappropriate expression of anti-diuretic hormone (SIADH).

In certain embodiments, the individual has a chronic condition and the individual has received therapy for oedema. In certain embodiments, the individual can have received therapy for oedema and/or received therapy for hypertension.

In certain embodiments, the individual has received diuretic therapy, e.g., therapy selected from one or more of administration of a diuretic selected from the group consisting of: a loop diuretic; a thiazide; a potassium-sparing diuretic, an osmotic diuretic, a carbonic anhydrase inhibitor, a Na/H exchanger antagonist; a selective vasopressin V2 antagonist, an arginine vasopressin receptor 2 antagonist; and an acidifying salt.

In certain embodiments, the methods disclosed herein can involve the further step of administering a further diuretic or anti-hypertensive compound to the individual.

In certain embodiments, the individual has abnormal kidney function, for example reduced FENa, FELi, urine volume or urinary flow rate. In certain embodiments, one or more of the preceding parameters is normal. For example, the individual can have early onset of a relevant condition, or have been prior treated with a diuretic or anti-hypertensive. Thus, in some embodiments, the individual may have one or more parameters indicative of normal kidney function.

In certain embodiments, the HPR or HPR signal peptide deletion variant may be as described above. In certain embodiments, the HPR or HPR signal peptide deletion variant is administered in the form of a composition suitable for intra-venous administration. In certain embodiments, the HPR or HPR signal peptide deletion variant may be administered to produce a plasma concentration of about 30-100 mg HPR per 70 kg individual. In certain embodiments, the HPR or HPR signal peptide deletion variant is administered from 1 to 3 times per day in an amount of about 30-100 mg.

As a result of the methods described herein, the individual can, in certain embodiments have an increased urinary flow rate, increased FENa, and increased FELi. Depending on the individual, these parameters can fall to below normal levels, hence requiring the repeated administration of HPR or HPR signal peptide deletion variant until the individual is stabilized.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1. Identification of Natriuretic Factor in Serum from Individuals with Alzheimer's Disease and Subarachnoid Hemorrhage

Background

The present study was designed to identify the natriuretic factor (NF) previously demonstrated in the plasma of patients with RSW by utilizing similar rat clearance methodology. We demonstrated natriuretic activity (NA) in sera of patients with identical clinical characteristics as patients who previously demonstrated NA in plasma, specifically subarachnoid hemorrhage (SAH) and advanced Alzheimer's disease (AD), whereas the serum 9 months after recovery of SAH and sera from 3 patients with a reset osmostat had no NA. The sera with NA were subjected to SWATH (Sequential Windowed Acquisition of All) that enabled the fold change (FC) ratio of proteins between sera with natriuretic activity and controls, calculated as the ratio of geometric means of the sample replicates. We found that haptoglobin related protein (HPR) satisfied the condition that the FCProtein ratio was >3.0 for both SAH and AD patients.

Methods

Human Studies

Internal Review Board for Human Research and the Institutional Animal Care and Use Committee of NYU Winthrop Hospital approved the studies. An informed signed consent was obtained from all study subjects including normal controls or healthcare proxy for cognitively impaired subjects. We have accumulated sufficient evidence, based on determinations of FEurate (3,11-17), to propose a new algorithm to approach patients with hyponatremic conditions. By utilizing this algorithm, we previously demonstrated natriuretic activity in the plasma of patients with advanced AD and SAH who had increased FEurate and largely normonatremia (20,21). The present studies were designed to extend these earlier studies to identify the natriuretic factor activity in both conditions. The clinical characteristics of the two patients involved in the current study were identical to patients studied in previous rat clearance studies (Table 1) (20,21).

Case Reports

SAH patient. A seventy-four-year-old female with a past medical history of hyperlipidemia, cataracts, laminectomy for lumbar disc herniation and neuropathic pain was admitted for acute onset of headache without loss of consciousness. Her blood pressure was 166/91 mmHg, pulse 61 beats per minute and temperature 97.7° F. Her neurologic examination was normal and the rest of her physical examination unremarkable. A non-contrast CT scan of brain showed diffuse subarachnoid hemorrhage with mild intraventricular hemorrhage. A CT angiogram showed a 6 mm bilobed anterior communicating artery aneurysm. She underwent successful coiling of the aneurysm the following day and had placement of an external ventricular drain for worsening hydrocephalus. The relevant laboratory results are shown in Table 1.

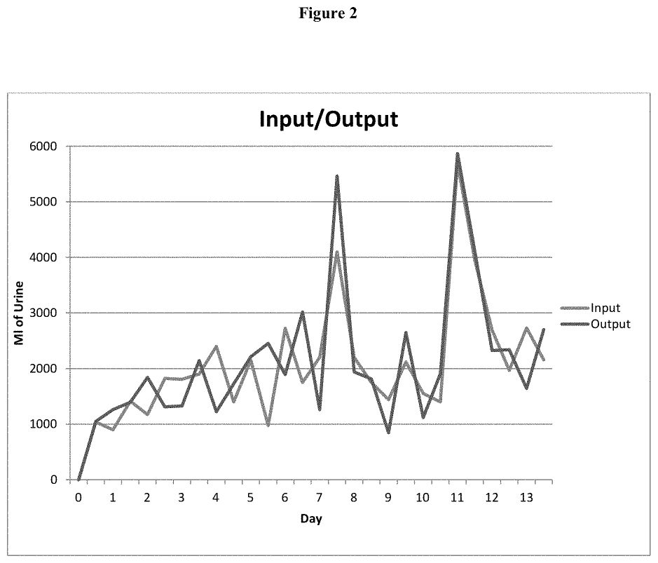

Her hospital course was marked by increased urine output that had to be matched by fluid resuscitation with isotonic saline. On days 2 and 5, fluid input lagged behind output when systolic blood pressures decreased to 98 and 93 mmHg, respectively (FIG. 2). Her urine output increased to levels exceeding 4 to 5 liters per day but hemodynamic stability was maintained by matching output with input (FIG. 2). On four occasions she required IV boluses of isotonic saline to maintain fluid balance and hemodynamic stability. Because the clinical course was consistent with a salt wasting syndrome, as is often noted in patients with SAH, she was recruited into our study on day 6. She did not develop hyponatremia at any time. On day 38, she complained of lethargy and was fully conversant by the time she was discharged to a subacute rehabilitation unit.

At the subacute rehabilitation unit, she received daily physical therapy for three months and then three times a week for another two months at home. She was fully self-sufficient and driving a car seven months after her intracranial bleed at which time her FEurate had normalized from a high 18.7 to 6.4% and her serum had no natriuretic activity. She offered a serum sample at this stage that we nominate as SAHpost.

AD patient. A seventy-four-year-old male with a history of seizure disorder, family history of AD in mother, aunt and uncle, gradual and insidious onset of cognitive decline over the previous two years was admitted to the hospital suffering from paranoid ideation and aggressive behavior towards his wife. A diagnosis of AD was made previously by neurologic and psychiatric assessments. On examination, he was alert, oriented to person, combative, non-cooperative and unable to identify the hospital, month or day of the week. A CT and an MRI of the head a year ago revealed mild cerebral volume loss. He responded well to a combination of Namenda, Seroquel, Aricep, Lexapro and Valproic Acid and discharged after three days. Pertinent laboratory results are listed in Table 1.

Storage of Serum Samples

Serum from human subjects was aliquoted into 0.55 ml lots and then stored at −80° C. until further use. Generally there was only one cycle of freeze/thawing performed prior to the use of the sample.

Haptoglobin (Hp) Phenotypes

For the determination of the haptoglobin serum concentration we used immunonephelometry on a Siemens BN II nephelometer and commercial antibodies (Siemens). The assay has been calibrated to the global WHO/DAP/IFCC standard. The Hp phenotypes (Hp1-1, Hp 2-1, Hp 2-2) in serum samples were determined by starch gel electrophoresis following the method described by Langlois et al (22). All measurements were performed at the Clinical Chemistry Laboratory University Hospital of Gent, Belgium.

Purified preparations of Hp1-1 and Hp2-2 were purchased from Athens Research (Athens, Ga.) and human recombinant HPR from Prospec (Israel). Hp depletion experiments of serum samples were performed using Hp specific antibody immuno-affinity chromatography columns. The antibody bound all three types of Hp that were absent on a Western blot. These experiments were performed by Athens Research (Athens, Ga.).

Renal clearance studies were performed by methods previously described with for a few modifications. Briefly, Male Sprague Dawley rats (Hilltop Lab Animals, Inc., Scottsdale, Pa.) weighing 350-450 grams were maintained in a standard animal facility with alternating dark-light conditions with free access to Envigo Tekland 8604 Rodent Diet and water up to the time of study. The rats were anesthetized with 1.3 mg/kg body weight of inactin (Sigma-Aldrich Corp., St Louis, Mo.). A tracheostomy was then performed, the jugular vein cannulated with PE50 polyethylene tubing followed by a cystotomy with a PE100 polyethylene tubing for urine collections. A half ml of isotonic saline, serum from a control or study patient or test substance dissolved in 0.5 ml isotonic saline was slowly infused into the jugular vein over two minutes followed by a priming dose of lithium chloride (60 mmol/L lithium chloride in isotonic saline) at a rate of 0.2 ml/min for three minutes followed by a constant infusion at a rate of 20 μl/min via a Harvard Compact Infusion pump up to the end of the study. Because proximal tubule sodium transport decreases abruptly with the infusion of saline, we eliminated the usual infusion of saline to replace surgical fluid losses. Twenty minutes after completion of the infusion of serum or test material, approximately 0.4 ml of blood was obtained from a cut tail. After 50 min equilibration period, timed urine collections were made at 30 min intervals for 2 to 2.5 h with additional blood being obtained at the end of the second urine collection and again at the end of the study. A 20 min equilibration period was also tested but gave identical results (data not shown).

Analytical Methods

Serum and urine creatinine, sodium, potassium, uric acid, phosphorus and lithium were determined by ADVIA chemistry system 1800, except for lithium in urine being determined by a Cole Parmer EW-02655-90 five element flame photometer. Serum and urine osmolalities were performed in a few serum and urine samples by freezing point depression in a Fiske model 210 micro osmometer.

SWATH Analysis

SWATH (Sequential Windowed Acquisition of All) MS is a data independent acquisition (DIA) method which aims to complement traditional mass spectrometry-based proteomics techniques such as shotgun and selected reaction monitoring (SRM) methods. In essence, it allows a complete and permanent recording of all fragment ions of the detectable peptide precursors present in a biological sample. All studies were performed at the Australian Proteome Analysis Facility, Sydney, Australia.

25 μL of plasma sample was diluted in 475 μL of 50 mM ammonium bicarbonate solution before reducing with dithiothreitol (5 mM DTT) and alkylating with iodoacetamide (10 mM IAA). One fifth of the sample (121 μL) was taken to digest with 10 μL trypsin (20 μg) for 16 h at 37° C. The digested sample was diluted in 0.1% formic acid to the final volume of 1375.5 μL and subjected to LC-MS/MS and LC-SWATH-MS analysis.

1D-Information dependent acquisition (IDA). The digested sample was diluted 1:1 with loading buffer. Ten μL of the digested and diluted sample was taken and subjected to 1D IDA nanoLC MS/MS analysis. The sample was diluted a further 1:1 and re-run to use as the seed file for SWATH.

Digested sample was diluted 1:1 with loading buffer. Ten μL of the diluted and digested sample was taken and transferred to HPLC vials for SWATH analysis.

A pool was prepared from 25 μg of each cleaned sample to perform high pH reverse phase fractionation on a HPLC column. Agilent 1260 quaternary HPLC system with Zorbax 300 Extend-C18 column (2.1 mm×150 mm, 3.5 μm, 300 Å column) was used for peptide high pH reverse phase HPLC fractionation. The buffer A was 5 mM ammonia solution (pH 10.5) and buffer B was 5 mM ammonia solution with 90% acetonitrile (pH 10.5). The dried digested sample was resuspended in loading buffer which was the same as the buffer A. After sample loading and washing with 97% buffer A for 10 minutes, buffer B concentration was increased from 3% to 30% for 55 minutes and then to 70% for 10 minutes and to 90% for another 5 minutes at a flow rate of 300 μL/min. The eluent of strong cation exchange (SCX) was collected every two minutes at the beginning of the gradient and every one-minute intervals for the rest of the gradient. All wells were dried; 50 μL loading buffer added. A total of 10 fractions were pooled from collected fractions (23-82 minutes), dried and resuspended in 50 μL of loading buffer. 10 μL of each fraction was transferred to vials for 2D IDA analysis.

5600 Triple time of flight (TOF) mass spectrometer (SCIEX) coupled with Eksigent Ultra nanoLC system (Eksigent) was used for MS data acquisition.

For 1D and 2D IDA nanoLC ESI MS/MS data, sample (10 μL) was injected onto a peptide trap (Halo C18, 150 μm×2 cm) for pre-concentration and desalted with 0.1% formic acid, 2% ACN, at 5 μL/min for three minutes. The peptide trap was then switched into line with the analytical column (Halo C18, 100 mm×150 μm, 160 Å, 2.7 μm.) Peptides were eluted from the column using linear solvent gradients, with steps, from mobile phase A: mobile phase B (98:2) to mobile phase A: mobile phase B (2:98) for 90 min, then to (65:35) where mobile phase A is 0.1% formic acid and mobile phase B is 99.9% ACN/0.1% formic acid at 600 nL/min over a 120 min period. After peptide elution, the column was cleaned with 98% buffer B for 10 min and then equilibrated with 98% buffer A for 15 minutes before the next sample injection. The reverse phase nanoLC eluent was subject to positive ion nanoflow electrospray analysis in an information dependent acquisition mode (IDA).

In the IDA mode a TOFMS survey scan was acquired (m/z 350-1500, 0.25 second), with the ten most intense multiply charged ions (2+-5+; counts >150) in the survey scan sequentially subjected to MS/MS analysis. MS/MS spectra were accumulated for 50 milliseconds in the mass range m/z 100-1500 with rolling collision energy.

SWATH data acquisition used identical nanoLC condition as IDA data acquisition. For SWATH MS, m/z window sizes were determined based on precursor m/z frequencies (m/z 400-1250) in previous IDA data (SWATH variable window acquisition, 60 windows in total). In SWATH mode, first a TOFMS survey scan was acquired (m/z 3501500, 0.05 sec) then the 60 predefined m/z ranges were sequentially subjected to MS/MS analysis. MS/MS spectra were accumulated for 90 milliseconds in the mass range m/z 350-1500 with rolling collision energy optimized for lowed m/z in m/z window+10%. SWATH data were acquired three times for each sample.

Data Processing

The LC-MS/MS data of the IDA runs were searched using ProteinPilot (v4.2) (AB Sciex) in thorough mode against human species from SwissProt 2016_02 (SwissProt_2016_02.fasta) [20,198 proteins]. A local plasma sample SWATH library was constructed using the database search results. Beside the local SWATH library, an extended library was made by merging an external plasma library (23) with the local library using an APAF developed program SwathXtend (24).

Local and extended libraries were used to extract SWATH peak areas separately using PeakView (SCIEX v2.1) with the following parameters: top 6 most intense fragments of each peptide were extracted from the SWATH data sets (75 ppm mass tolerance, 10 min retention time window). Shared and modified peptides were excluded. After data processing, peptides (maximum 100 peptides per protein) with confidence >99% and false discovery rate (FDR) 1% (based on chromatographic feature after fragment extraction) were used for quantitation. The extracted SWATH protein peak areas were further analyzed by APAF in-house program. The protein peaks were normalized to the total peak area for each run and subjected to T-Test to compare relative protein peak area between the sample group. Protein T-Test with a P-value smaller than 0.05 and a fold change larger than 1.5 were highlighted. Details of the analysis have been described previously (24).

Two approaches were evaluated for determining differentially expressed proteins: the simple approach of working with the protein level quantitation only and the second working with the peptide level quantitation separately for each peptide. For the protein level approach featured in the Results section, differential expression was assessed by a two-sample t-test or ANOVA of the log transformed normalized protein peak areas. Natural logs (base e) were used throughout. The fold change (FC) ratio between any two conditions was calculated as the ratio of geometric means of the sample replicates, which corresponds to calculating the normal arithmetic ratio of log-transformed areas and back-transforming.

For the peptide level approach, fold changes between the two categories were determined for each peptide separately as the ratio of average abundances in the two different categories. Then, differential expression was assessed by a one sample t-test of all log-transformed peptide fold changes corresponding to a particular protein. The advantage of using the peptide approach is that peptides of lower intensity can contribute without being dwarfed by the high intensity peptides; the disadvantage is that at least two different fold changes, hence two different peptides, are necessary for the calculation of the one sample t-test in this scenario, hence single peptide proteins cannot be considered as differentially expressed. The protein-level fold change was calculated as the geometric average of individual peptide-level fold changes.

Statistics

Statistical significance for in vivo rat studies was determined by a 2-tailed Student's T-Test, where P<0.05 was considered statistically significant.

Results

Diagnosis of RSW

The clinical diagnosis of RSW in our patient (female) with SAH was collectively based on the following data: there is general agreement that RSW occurs very commonly in hyponatremic and normonatremic patients with SAH, 89% and 67% respectively, with data from one study showing volume depletion using gold standard radio isotope dilution methods (25). The clinical course of the patient after sustaining SAH is typical of RSW, with excretion of large volumes of urine that required increased volumes of saline to maintain hemodynamic stability (FIG. 2). Increased FEurate has been amply reported in patients with SIADH and RSW when hyponatremic, but FEurate is uniquely different when normonatremic, being normal in SIADH and persistently increased in RSW (11). The high FEurate in the presence of normonatremia in the patient with SAH is consistent with RSW, as is the increased FEphosphate, which has been demonstrated in RSW, but not SIADH (11). Natriuretic activity has been demonstrated in plasma and urine of largely normonatremic neurosurgical patients with increased FEurate (20,26). The increased FEurate in the normonatremic patient with SAH and the increased urine output requiring large volumes of saline to maintain hemodynamic stability are consistent with RSW.

The clinical diagnosis of RSW in AD is less defined than that for SAH and neurosurgical diseases. It is being introduced as a new observation that is supported by having increased FEurate with normonatremia and presence of natriuretic activity in their plasma (21).

The present studies were designed to identify the natriuretic factor in both AD and SAH. The clinical characteristics of both patients were identical to patients studied in previous rat clearance studies (Table 1) (20,21).

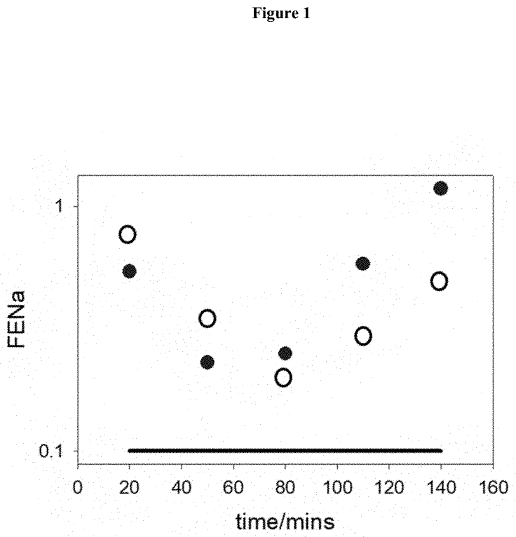

The potency of serum from patients with RSW is evident when the serum is introduced into control rats. Material with diuretic activity is introduced as an intravenous bolus of 0.5 ml into the jugular vein. The diuretic effect in the kidney will be generated by the first blush of diuresis as the bolus hits the kidney for the first time. Subsequent passes will likely have no effect as the bolus will be severely diluted in the circulation and the activity is markedly concentration dependent. Variation will be associated with the level of dilution of the bolus in its first passage by the kidney. In steady state infusion, the variation is considerably less. With both serum samples from SAH and AD patients we can mimic the profound features of RSW including increases in FENa, FELi and UFR (FIG. 1 and Table 2). The induction of the RSW effect is relatively rapid and can be maintained for at least 170 min. The RSW effect is accompanied by a statistically significant increase in FENa, FELi and UFR. FEurate did not change (Table 2). It appears that the major site of inhibition of Na transport resides in the proximal tubule because lithium, which is also inhibited, is transported exclusively in the proximal tubule.

Remarkably, the similar kinetics of action and the relative change seen to occur suggest that the factor causing these RSW effects is the same in both SAH and AD. The reasons for the relatively high degree of variation are unclear, although we have observed some loss of RSW activity in the serum preparations with storage over one year, especially after thawing and refreezing several times (unpublished data).

SWATH analysis with an extended library may detect up to 650 proteins in a serum sample. We have explored the data to identify those genes that are significantly upregulated in both SAH and AD (Table 3). The FCProtein parameter will give a semi-quantitative estimate of the relative change of a particular protein versus a control sample where the agreement to analytically measured concentration is relatively good. So for SAH vs control, the ratio of analytical Hp concentration is 3.7 (Table 4), whereas the FCProtein is 5.86. A similar calculation would demonstrate the Hp ratio, as the AD sample would be 1.5 compared to FCProtein value of 1.5.

Maximal changes in the FCProtein ratio for both SAH and AD patients as compared to controls, are seen with HPR (Table 3). Hp has been included, as the SWATH analysis does not distinguish between the various phenotypes of Hp. So rather than an upregulation of the Hp, there may have been a change in the phenotype with the disease process. In exploring this idea we identified a major finding that depletion of Hp from the serum sample from either the SAH patient or the AD patient results in removal of RSW activity (Table 5). SWATH analysis of the SAH depleted serum indicated an expected FCProtein of 48.8 for Hp (control vs depleted serum). However, it was not immediately apparent as to which Hp phenotype was responsible for the RSW activity as both SAH and AD patients expressed quite different phenotypes (Table 4); 2-1 and 1-1 respectively. The conclusions were further confounded by the discovery that lowering the Hp 2-1 concentration by 50% in SAH post (Table 4), did not change the Hp phenotype, but still resulted in removal of RSW activity, indicating that the natriuretic factor may not be Hp.

Another way that we examined the SWATH data was to investigate the maximal disproportional change in FCProtein of the SAH patient vs SAH patient post. The other candidate natriuretic factor proteins did not change significantly and were essentially in the same ratio as Hp (Table 3).

What was surprising is that the SWATH analysis indicated the FCProtein for HPR (a candidate natriuretic factor, Table 3) was 111.3 for the Hp depleted serum from the SAH patient. This clearly demonstrated that the immuno-affinity column used for Hp depletion was not specific for Hp but it also bound and depleted HPR. The RSW activities of Hp depleted samples and other proteins confirmed that HPR was the natriuretic factor present in both SAH and AD serum (Table 5).

Discussion

The present studies successfully identify the natriuretic factor that was previously demonstrated in the plasma of patients with neurosurgical and Alzheimer's diseases by an identical in vivo rat renal clearance method where competing variables are interactive as compared to a more controlled in vitro system (20,21). SWATH analysis of sera of control and sera from patients with SAH and AD with natriuretic activity identified several candidate proteins that might be the factor that induces RSW (Table 3). Identification of HPR as the factor that induces RSW was accomplished by several approaches that started with the SWATH analysis which favored haptoglobins as the most probable class of proteins. The sera from the patients SAH and AD with natriuretic activity had total Hp levels of 467 and 196 mg/dL respectively, and the serum from the patient with SAH, after recovering from her SAH eight months after sustaining SAH, had no natriuretic activity despite having elevated levels of Hp 2-1 level at 258 mg/dL. Depletion of Hp by immunodepletion methods demonstrated absence of Hp by Western blotting in both active sera from SAH and AD, and identification of the specific isoforms of Hp revealed the SAH serum to contain Hp 2-1, as compared to Hp 1-1 in AD serum. The serum with no natriuretic activity from the recovered patient with SAH continued to have high Hp 2-1 levels, suggesting that Hp 2-1 was not the natriuretic factor. Moreover, infusion rats with Hp 1-1 and 2-2 had no natriuretic activity at different doses (Table 5). Finally, infusion of recombinant HPR induced increases in urine volume and increased fractional excretions of sodium, and lithium, similar to those induced by active SAH and AD sera.

HPR is a plasma protein with 91% sequence homology to Hp1-1. It is synthesized as an approximate 45 kDa protein that binds efficiently to hemoglobin (27) and mediates trypanosome lytic factor binding to trypanosomes (28). The human HPR gene is only located 2.2 kilobases downstream of the Hp gene. One may expect that there is a relationship between the circulating levels of HPR and Hp, although their relative expression is markedly different. Normal human serum with HPR levels of 30-40 μg/ml (28) (reported to be in 49 μg/ml (29) in Caucasian children) compares to levels of Hp of 0.9-3 mg/ml (28). There are clearly a number of factors affecting the ratio of FCHp/FCHPR, particularly at the level of active material presenting at the the proximal tubule. Glomerular filtration of a 45 kDa protein will be relatively high (30), as compared to the higher molecular weight Hp (Hp 1-1 86 kDa, Hp 2-1 86-300 kDa, Hp2-2 170-900 kDa (33)). Further divergence of FCHp/FCHPR will occur in RSW states. The FCHPR becomes >5.5 for both SAH and AD (Table 3), whereas while there is an increase in Hp for SAH, there is little change in AD. We do note that the Hp 2-2 phenotype has been associated with SAH (34), whereas we have demonstrated an upregulation of Hp 2-1 in SAH (Table 3). These results establish the important diagnostic value of the levels of HPR in diagnosing RSW in both AD and SAH.

It appears that the major site of HPR inhibition of solute transport resides in the proximal tubule because lithium is transported exclusively in the proximal tubule (31).

The absence of glycosuria (data not shown) does not meet the criteria for a full-fledged Fanconi syndrome, but future studies should investigate the effect of HPR on other features of the Fanconi syndrome, such as inhibiting bicarbonate and amino acid transport.

While HPR can be effectively utilized clinically as a biomarker to identify hyponatremic or normonatremic patients with RSW, treatment with salt and water supplementation would overcome the pathophysiologic deficiency, but can significantly alter quality of life by having to urinate frequently, especially nocturia every 1-2 h. The use of an inhibitor of the natriuretic factor would eliminate the increased frequency of urination and nocturia induced by salt and water supplementation.

TABLE 1
Clinical features of the SAH and AD patients.

| | | | | AD | | |
|---|---|---|---|---|---|---|
| SAH | | | | patient | | |
| patient | Serum | Urine | FE | Serum | Urine | FE |
| Na | 139 | 202 | 1.89 | 142 | 111 | 0.35 |
| K | 5.1 | 28 | 10.4 | 4.1 | 56.8 | 6.23 |
| Cl | 105 | | | 106 | | |
| CO2 | 31 | | | 33.1 | | |
| Uric acid | 2.6 | 25.7 | 18.7 | 6.5 | 63.2 | |
| PO4 | 3.1 | 21.4 | 21.4 | 3.7 | 88.6 | 10.8 |
| Cr | 0.5 | 53 | | 0.8 | 178 | 4.37 |
| Ca | | | | 9.1 | | |
| BUN | 16 | | | 10 | | |

TABLE 2
Rat renal parameters resulting from infusion of serum samples from an SAH patient and AD patient.

| | | TTest | | TTest | | TTest | | TTest |
|---|---|---|---|---|---|---|---|---|
| | | vs | | vs | | vs | UFR/ | vs |
| Patient | FENa | control | FELI | control | FEUrate | control | μl/min | control |
| AD | 0.52 ± 0.6 | 0.007 | 52.2 ± 21.5 | 0.006 | 21.9 ± 5.7 | 0.22 | 13.7 ± 7.4 | 0.002 |
| SAH | 0.67 ± 0.5 | 0.021 | 46.1 ± 17.8 | 0.006 | 19.2 ± 5.4 | 0.79 | 20.4 ± 16.0 | 0.024 |
| SAHpost | 0.21 ± 0.12 | | 23.9 ± 6.2 | | 20.5 ± 4.5 | | 8.2 ± 3.4 | |
| Controls | 0.10 ± 0.05 | | 24.4 ± 8.3 | | 18.5 ± 4.5 | | 6.1 ± 2.2 | |

The averages were taken over the time period 50-140 min where the time represents the start of a 30 min collection. The number of serum samples used is represented by n. Data for AD patient (n=5) was compared to healthy controls (n=13) whereas the data for the SAH patient was obtained by comparing data for serum obtained during the hyponatremic episode (n=4) as compared to the same patient (nine months later) where the patient was normal (n=4).

TABLE 3
Featured genes that are significantly
upregulated in neurological disease.

| Gene | | FCProteins (P-value) | | |
|---|---|---|---|---|
| Accession | | SAH | SAH vs | AD vs |
| No | Protein | vs control | SAH post | control |
| P00738 | Haptoglobin | 5.86(<0.001) | 2.09(<0.001) | 1.5(<0.001) |
| P00739 | Haptoglobin | 5.42(<0.001) | 1.97(<0.001) | 6.1(<0.001) |
| | related protein | | | |

FCProtein data from SAH vs control, SAH vs SAHpost and AD vs control. FCProtein represents the fold-change ratio.

TABLE 4
Characterization of the Hp phenotype in different patient samples.

| | | | Hp | |
|---|---|---|---|---|
| | Hp | | Phenotype | HPR serum |
| | concentration/ | Hp | mol wt | concentration |
| Patient | mg/dl | phenotype | range/kDa | range/mg/dl |
| AD | 196 | 1-1 | 86 | 74.7 |
| SAH | 467 | 2-1 | 100-130 | 32-42 |
| SAH post | 258 | 2-1 | 100-130 | not tested |
| Control | 126 | 2-2 | 190-500 | 3-8 |

TABLE 5
RSW activity as measured by FENa for various exogenous
preparations introduced into the rat model

| Protein preparation | RSW activity |
|---|---|
| SAH serum vs Hp immuno-depleted SAH serum | 0.82 vs 0.38 |
| AD serum vs Hp immune-depleted AD serum | 0.61 vs 0.13 |
| Hp 1-1 (range 0.5-4.0 mg/ml) | 0.1 |
| Hp 2-2 (range 0.5-4.0 mg/ml) | 0.16 |
| Recombinant haptoglobin related protein (HPR) | 0.31 ± 0.15 |
| containing a signal peptide (212 μg/ml) | (p < 0.0001 vs control) |

The estimated concentrations of serum HPR are shown in Table 4. There is clearly a significant increase in HPR levels with individual A and B.

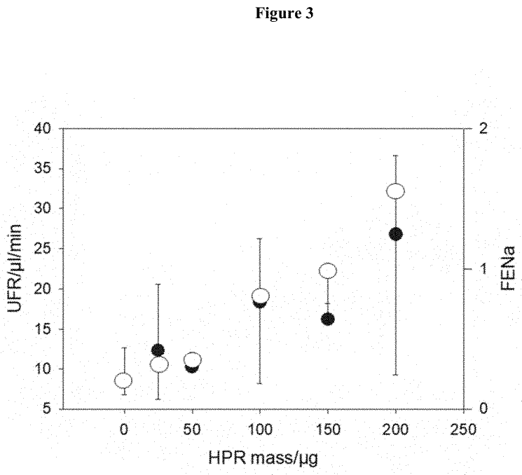

In all the studies associated with mass spectrometry of purified urine sample and SWATH analysis of serum samples the signal peptide of HPR was not detected. The diuretic activity of purified HPR without the signal peptide (aas1-18 from N-terminus) was then determined. This material exhibited significant dose dependent diuretic activity when introduced intravenously in healthy rats. In a similar fashion to clinical serum samples, the introduction of HPR results in a statistically significant increase in FENa and UFR (FIG. 3).

Polymorphisms in the haptoglobin gene cluster have been described. HPR is a primate specific plasma protein with 91% sequence homology to Hp1-1. It is synthesized as an approximate 45 kDa protein. HPR binds efficiently to hemoglobin and mediates trypanosome lytic factor binding to trypanosomes. The human HPR gene is only located 2.2 kilobases downstream of the Hp gene. One may expect that there is a relationship between the circulating levels of HPR and Hp and this has been demonstrated in children from Gabon. In fact, that while their relative expression is markedly different, it has been demonstrated that HPR levels were more closely correlated with Hp1-1 and 2-1 levels rather than 2-2. Normal human serum with HPR levels of 30-40 μg/ml (reported to be in 49 μg/ml in Caucasian children) compares to levels of Hp of 0.9-3 mg/ml.

The profound increase in UFR in FIG. 3 demonstrates the potent diuretic properties of HPR acting at the proximal tubule. Thus, an important clinical application of HPR comes from its use as a diuretic, especially to treat congestive heart failure.

### REFERENCES

1. Peters J P, Welt L G, Sims E A, Orloff J, Needham J. A salt-wasting syndrome associated with cerebral disease. Trans Assoc Am Physicians 1950; 63: 57-64
2. Schwartz W B, Bennett W, Curelop S, Bartter F C. A syndrome of renal sodium loss and hyponatremia probably resulting from inappropriate secretion of antidiuretic hormone. Am J Med 1957; 23: 529-542
3. Maesaka J K, Imbriano L, Shirazian S, Miyawaki N. Complexity of differentiating cerebral-renal salt wasting from SIADH, emerging importance of determining fractional urate excretion. In: Vijayakumar S, editor. Novel insights on chronic kidney disease, acute kidney injury and polycystic kidney disease. Croatia: InTech 2012; 41-66
4. Maesaka J K, Miyawaki N, Palaia T, Fishbane S, Durham J H. Renal salt wasting without cerebral disease: diagnostic value of urate determinations in hyponatremia.

Kidney Int 2007; 71: 822-826
5. Bitew S, Imbriano L, Miyawaki N, Fishbane S, Maesaka J K. More on renal salt wasting without cerebral disease: response to saline infusion. Clin J Am Soc Nephrol 2009; 4: 309-315
6. Wijdicks E F, Vermeulen M, ten Haaf J A, Hijdra A, Bakker W H, van Gijn J. Volume depletion and natriuresis in patients with a ruptured intracranial aneurysm. Ann Neurol 1985; 18: 211-216
7. Schrier R W. Does ‘asymptomatic hyponatremia’ exist? Nat Rev Nephrol 2010; 6: 185
8. Renneboog B, Musch W, Vandemergel X, Manto M U, Decaux G. Mild chronic hyponatremia is associated with falls, unsteadiness, and attention deficits. Am J Med 2006; 119: 71.e1-71.e8
9. Gankam Kengne F, Andres C, Sattar L, Melot C, Decaux G. Mild hyponatremia and risk of fracture in the ambulatory elderly. QJM 2008; 101: 583-588
10. Chung H M, Kluge R, Schrier R W, Anderson R J. Clinical assessment of extracellular fluid volume in hyponatremia. Am J Med 1987; 83: 905-908
11. Maesaka J K, Imbriano L J, Miyawaki N. Application of established pathophysiologic processes brings greater clarity to diagnosis and treatment of hyponatremia. World J Nephrol 2017; 6: 59-71
12. Maesaka J K, Batuman V, Yudd M, Salem M, Sved A F, Venkatesan J. Hyponatremia and hypouricemia: differentiation from SIADH. Clin Nephrol 1990; 33: 174-178
13. Maesaka J K, Cusano A J, Thies H L, Siegal F P, Dreisbach A W. Hypouricemia in acquired immunodeficiency syndrome. Am J Kidney Dis 1990; 15: 252-257
14. Maesaka J K, Venkatesan J, Piccione J M, Decker R, Dreisbach A W, Wetherington J D. Abnormal urate transport in patients with intracranial disease. Am J Kidney Dis 1992; 19: 10-15
15. Imbriano L J, Mattana J, Drakakis J, Maesaka J K. Identifying different causes of hyponatremia with fractional excretion of uric acid. Am J Med Sci 2016; 352: 385-390
16. Imbriano L J, Ilamathi E, Ali N M, Miyawaki N, Maesaka J K. Normal fractional urate excretion identifies hyponatremic patients with reset osmostat. J Nephrol 2012; 25: 833-838
17. Maesaka J K, Imbriano L J, Ali N M, Ilamathi E. Is it cerebral or renal salt wasting? Kidney Int 2009; 76: 934-938
18. Wijdicks E F, Vermeulen M, Hijdra A, van Gijn J. Hyponatremia and cerebral infarction in patients with ruptured intracranial aneurysms: is fluid restriction harmful? Ann Neurol 1985; 17: 137-140
19. Gutierrez O M, Lin H Y. Refractory hyponatremia. Kidney Int 2007; 71: 79-82
20. Maesaka J K, Venkatesan J, Piccione J M, Decker R. Dreisbach A W, Wetherington J. Plasma natriuretic factor(s) in patients with intracranial disease, renal salt wasting and hyperuricosuria. Life Sci 1993; 52:1875-1882
21. Maesaka J K, Wolf-Klein G, Piccione J M, Ma C M. Hyporuicemia, abnormal renal tubular urate transport, and plasma natriuretic factor(s) in patients with Alzheimer's disease. J Am Geriatr Soc 1993; 41: 501-506
22. Langlois M R, Martin M E, Boelaert J R, Beaumont C, Taes Y E, De Buyzere M L, Bernard D R, Neels H M, Delanghe J R. The haptoglobin 2-2 phenotype affects serum markers of iron status in healthy males. Clin Chem 2000, 46:1619-1625
23. Liu Y, Buil A, Collins B C, Gillet L C, Blum L C, Cheng L Y, Vitek O, Mouritsen J, Lachance G, Spector T D, Dermitzakis E T, Aebersold R. Quantitative variability of 342 plasma proteins in a human twin population. Mol Syst Biol 2015; 4: 786.
24. Wu J X, Song X, Pascovici D, Zaw T, Care N, Krisp C, Molloy M P. SWATH mass spectrometry performance using extended peptide MS/MS assay libraries. Mol Cell Proteomics 2016; 15: 2501-2514
25. Wijdicks E F, Vermeulen M, Haaf J A, Hijdra A, Bakker W H, van Gijn J. Volume depletion and natriuresis in patients with a ruptured intracranial aneurysm. Ann Neurol 1985; 18: 211-216
26. Youmans S J, Fein M R, Wirkowski E, Maesaka J K. Demonstration of natriuretic activity in urine of neurosurgical patients with renal salt wasting. F1000Research 2013; 2:126
27. Nielsen M J, Petersen S V, Jacobsen C, Oxvig C, Rees D, Møller H J, Moestrup S K. Haptoglobin-related protein is a high-affinity hemoglobin-binding plasma protein. Blood 2006; 108: 2846-2849
28. Drain J, Bishop J R, Hajduk S L. Haptoglobin-related protein mediates trypanosome lytic factor binding to trypanosomes. J Biol Chem 2001; 276: 30254-30260
29. Imrie H J, Fowkes F J, Migot-Nabias F, Luty A J, Deloron P, Hajduk S L, Day K P. Individual variation in levels of haptoglobin-related protein in children from Gabon. PLoS One 2012; 7:e49816
30. Comper W D, Russo L M, Vuchkova J. Are filtered plasma proteins processed in the same way by the kidney? J Theor Biol 2016; 410: 18-24
31. Hayslett J P, Kashgarian M. A micropuncture study of the renal handling of lithium. Pflugers Arch 1979; 380: 159-163
32. Abuelo J G. Normotensive ischemic acute renal failure. N Eng J Med 2007; 357: 797-805
33. Langlois M R, Delanghe J R. Biological and clinical significance of haptoglobin polymorphism in humans. Clin Chem 1996; 42:1589-1600
34. Murthy S B, Caplan J, Levy A P, Pradilla G, Moradiya Y, Schneider E B, Shalom H, Ziai, W C, Tamargo R J, Nyquist P A. Haptoglobin 2-2 genotype is associated with cerebral salt wasting syndrome in aneurysmal subarachnoid hemorrhage. Neurosurgery 2016; 78: 71-76
35. Ellison D H, Berl T. The syndrome of inappropriate antidiuresis. N Engl J Med 2007, 356, 2064-2072
36. Corona G, Giuliani C, Parenti G, Norello D, Verbalis J G, Forti G, Maggi M, Peri A. Moderate hyponatremia is associated with increased risk of mortality: Evidence from a meta-analysis. Plus-one 2013; 8(12):e80451
37. Berl T, Quittnat-Pelletier F, Verbalis J G, Schrier R W, Bichet D G, Ouyang J, & Czerwiec F S; SALTWATER Investigators. (2010) Oral tolvaptan is safe and effective in chronic hyponatremia. J Am Soc Nephrol, 21, 705-712
38. Decaux G. Is asymptomatic hyponatremia really asymptomatic? Am J Med 2006, 119 (7 Suppl 1) S79-S82.
39. Hoorn E J, Van Der Lubbe N, Zietse R. SIADH and hyponatremia: why does it matter? NDT plus 2009, (suppl 3), pp. iii5-iii11
40. Arieff A I, Llach F, & Massry S G. (1976) Neurological manifestations and morbidity of hyponatremia: correlation with brain water and electrolytes. Medicine, 1976, (Baltimore), 55(2), 121-129
41. Verbalis J G, Barsony J, Sugimura Y, Tian Y, Adams D J, Carter E A, Resnick H E. Hyponatremia-induced osteoporosis. J Bone Min Metab, 2010, 25, 554-563
42. Maeska, J K, Imbriano L J, Miyawaki N. High prevalence of renal salt wasting without cerebral disease as a cause of hyponatremia in general medical wards. Am J Med Sci 2018 (in press).
43. Smith A B, Esko J D, and Hajduk S L Killing of Trypanosomes by the human haptoglobin-related protein. Science 1995 268, 284-286.

Sequence listing

SEQ ID No: 1 (HPR)(from >sp|P00739|1-348)
MSDLGAVISLLLWGRQLFALYSGNDVTDISDDRFPKPPEIANGYVEHLFR
YQCKNYYRLRTEGDGVYTLNDKKQWINKAVGDKLPECEAVCGKPKNPANP
VQRILGGHLDAKGSFPWQAKMVSHHNLTTGATLINEQWLLTTAKNLFLNH
SENATAKDIAPTLTLYVGKKQLVEIEKVVLHPNYHQVDIGLIKLKQKVLV
NERVMPICLPSKNYAEVGRVGYVSGWGQSDNFKLTDHLKYVMLPVADQYD
CITHYEGSTCPKWKAPKSPVGVQPILNEHTFCVGMSKYQEDTCYGDAGSA
FAVHDLEEDTWYAAGILSFDKSCAVAEYGVYVKVTSIQHWVQKTIAEN
SEQ ID No: 2 (α subunit of HPR)
LYSGNDVTDISDDRFPKPPEIANGYVEHLFRYQCKNYYRLRTEGDGVYTL
NDKKQWINKAVGDKLPECEAVCGKPKNPANPVQR
SEQ ID No: 3 (β subunit of HPR)
ILGGHLDAKGSFPWQAKMVSHHNLTTGATLINEQWLLTTAKNLFLNHSEN
ATAKDIAPTLTLYVGKKQLVEIEKVVLHPNYHQVDIGLIKLKQKVLVNER
VMPICLPSKNYAEVGRVGYVSGWGQSDNFKLTDHLKYVMLPVADQYDCIT
HYEGSTCPKWKAPKSPVGVQPILNEHTFCVGMSKYQEDTCYGDAGSAFAV
HDLEEDTWYAAGILSFDKSCAVAEYGVYVKVTSIQHWVQKTIAEN
SEQ ID No: 4 (HP)(from >sp|P00738|19-406)
VDSGNDVTDIADDGCPKPPEIAHGYVEHSVRYQCKNYYKLRTEGDGVYTL
NDKKQWINKAVGDKLPECEADDGCPKPPEIAHGYVEHSVRYQCKNYYKLR
TEGDGVYTLNNEKQWINKAVGDKLPECEAVCGKPKNPANPVQRILGGHLD
AKGSFPWQAKMVSHHNLTTGATLINEQWLLTTAKNLFLNHSENATAKDIA
PTLTLYVGKKQLVEIEKVVLHPNYSQVDIGLIKLKQKVSVNERVMPICLP
SKDYAEVGRVGYVSGWGRNANFKFTDHLKYVMLPVADQDQCIRHYEGSTV
PEKKTPKSPVGVQPILNEHTFCAGMSKYQEDTCYGDAGSAFAVHDLEEDT
WYATGILSFDKSCAVAEYGVYVKVTSIQDWVQKTIAEN
SEQ ID No: 5 (α subunit of HP)(from >sp|P00738|
19-160)
VDSGNDVTDIADDGCPKPPEIAHGYVEHSVRYQCKNYYKLRTEGDGVYTL
NDKKQWINKAVGDKLPECEADDGCPKPPEIAHGYVEHSVRYQCKNYYKLR
TEGDGVYTLNNEKQWINKAVGDKLPECEAVCGKPKNPANPVQ
SEQ ID No: 6 (β subunit of HP)(from >sp|P00738|
162-406
ILGGHLDAKGSFPWQAKMVSHHNLTTGATLINEQWLLTTAKNLFLNHSEN
ATAKDIAPTLTLYVGKKQLVEIEKVVLHPNYSQVDIGLIKLKQKVSVNER
VMPICLPSKDYAEVGRVGYVSGWGRNANFKFTDHLKYVMLPVADQDQCIR
HYEGSTVPEKKTPKSPVGVQPILNEHTFCAGMSKYQEDTCYGDAGSAFAV
HDLEEDTWYATGILSFDKSCAVAEYGVYVKVTSIQDWVQKTIAEN
SEQ ID No: 7
MSDLGAVISLLLWGRQLFA
SEQ ID No: 8
TEGDGVYTLNDKK
SEQ ID No: 9
LPECEAVCGKPK
SEQ ID No: 10
VMPICLPSK
SEQ ID No: 11
AVGDKLPECEAVCGKPKN
SEQ ID No: 12
DIAPTLTLYVGK
SEQ ID No: 13
SCAVAEYGVYVK
SEQ ID No: 14
ALYSGNDVTDISDDRF
SEQ ID No: 15
NGYVEHLFRYQCKNYYR
SEQ ID No: 16
NQVDIGLIKLKQKVL
SEQ ID No: 17
NYAEVGRVGYVSGWGQSDNFKL
SEQ ID No: 18
YDCITHYEGSTCPKWKAP
SEQ ID No: 19
FCVGM
SEQ ID No: 20
YAAGILS

The invention claimed is:

1. A method for treating an individual for Renal Salt Wasting (RSW) or for one or more symptoms thereof comprising:
(a) providing or having provided a control describing the amount of serum or plasma Haptoglobin related protein (HPR) in a normal individual;
(b) assessing or having assessed a serum or plasma test sample obtained from an individual for whom RSW or one more symptoms thereof is to be treated, to determine the amount of HPR contained in the test sample;
(c) comparing or having compared the amount of HPR in the test sample with the control;
(d) treating the individual with either (i) salt and water or (ii) an HPR antagonist if the amount of serum or plasma HPR in the test sample is more than the amount of serum or plasma HPR in the control; and
thereby treating the individual for RSW or one or more symptoms thereof.

2. The method of claim 1 wherein the individual:
a. is non-oedematous;
b. is hypouricemic;
c. has an increased fractional excretion of uric acid (FEUA);
d. forms concentrated urine;
e. has a high urine sodium concentration;
f. has a symptom selecting from the group consisting of nausea, malaise, lethargy, confusion, decreased cognitive function or consciousness and headache;
g. has an acute symptom of RSW
h. has a chronic symptom of RSW; and/or
i. is normonatremic at the time of treatment

3. The method of claim 1 wherein the HPR antagonist is an anti-HPR antibody.

4. A method for treating an individual for the syndrome of inappropriate anti-diuretic hormone secretion (SIADH) or for one or more symptoms thereof comprising:
(a) providing or having provided a control describing the amount of serum or plasma Haptoglobin related protein (HPR) in a normal individual;
(b) assessing or having assessed a serum or plasma test sample obtained from an individual for whom SIADH or one more symptoms thereof is to be treated, to determine the amount of HPR contained in the test sample;
(c) comparing or having compared the amount of serum or plasma HPR in the test sample with the control;
(d) treating the individual with a vasopressin receptor antagonist where the amount of serum or plasma HPR in the test sample is the same as or less than the amount of serum or plasma HPR in the control; and
thereby treating the individual for hyponatremia or one or more symptoms thereof.

5. The method of claim 4 wherein the individual:
a. is non-oedematous;
b. is hypouricemic;
c. has an increased fractional excretion of uric acid (FEUA);
d. forms concentrated urine;
e. has a high urine sodium concentration;
f. has a symptom selecting from the group consisting of nausea, malaise, lethargy, confusion, decreased cognitive function or consciousness and headache;
g. has an acute symptom of RSW; and/or
h. has a chronic symptom of RSW.

6. The method of claim 5 wherein the vasopressin receptor antagonist is a vaptan.

7. The method of claim 6 wherein the vaptan is selected from the group consisting of: conivaptan, tolvaptan, stavaptan, lixivapatan, and mozavaptan.

8. A method for:
a. inducing diuresis in an individual;
b. increasing fractional excretion of sodium (FENa) in an individual; or
c. increasing urinary flow rate in an individual;
comprising administering haptoglobin related protein (HPR) or HPR signal peptide deletion variant to an individual in whom diuresis is to be induced, FENa is to be increased, or urinary flow rate is to be increased, thereby inducing diuresis in the individual; increasing FENa in the individual; or increasing urinary flow rate in the individual.

9. The method of claim 8 wherein the individual has oedema.

10. The method of claim 8 wherein the individual has a condition selected from the group consisting of nephrotic syndrome, chronic kidney disease, congestive heart failure and liver cirrhosis.

11. The method of claim 8 wherein the individual has: received therapy for oedema; received therapy for hypertension; and/or received diuretic therapy.

12. The method of claim 8 wherein the individual has been administered a diuretic selected from the group consisting of: a loop diuretic; a thiazide; a potassium-sparing diuretic, an osmotic diuretic, a carbonic anhydrase inhibitor, a Na/H exchanger antagonist; a selective vasopressin V2 antagonist, an arginine vasopressin receptor 2 antagonist; and an acidifying salt.

13. The method of claim 8 comprising the further step of administering a further diuretic or anti-hypertensive compound to the individual.

14. The method of claim 8 wherein the individual has normal kidney function.

15. The method of claim 8 wherein the HPR or HPR signal peptide deletion variant induces diuresis at the proximal tubule.

16. The method of claim 8 wherein the HPR or HPR signal peptide deletion variant is administered in the form of a composition suitable for intra-venous administration.

17. The method of claim 8 wherein the HPR or HPR signal peptide deletion variant is administered to produce a plasma concentration of about 30-100 mg HPR per 70 kg individual.

18. The method of claim 8 wherein the HPR or HPR signal peptide deletion variant is administered from 1 to 3 times per day in an amount of about 30-100 mg.

19. A composition suitable for intravenous administration comprising HPR or HPR signal peptide deletion variant as an active diuretic principle and a carrier, excipient or solvent suitable for intravenous administration.

20. The composition of claim 19 wherein the HPR or HPR signal peptide deletion variant induces diuresis at the proximal tubule.


SEQUENCE LISTING (20 sequences)

SEQ ID NO: 1 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 2 - protein, 84 aa, Homo sapiens
LYSGNDVTDISDDRFPKPPEIANGYVEHLFRYQCKNYYRLRTEGDGVYTLNDKKQWINKAVGDKLPECEAVCGKPKNPANPVQR

SEQ ID NO: 3 - protein, 245 aa, Homo sapiens
ILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 4 - protein, 388 aa, Homo sapiens
VDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 5 - protein, 142 aa, Homo sapiens
VDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQ

SEQ ID NO: 6 - protein, 245 aa, Homo sapiens
ILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 7 - protein, 19 aa, Homo sapiens
MSDLGAVISLLLWGRQLFA

SEQ ID NO: 8 - protein, 13 aa, Homo sapiens
TEGDGVYTLNDKK

SEQ ID NO: 9 - protein, 12 aa, Homo sapiens
LPECEAVCGKPK

SEQ ID NO: 10 - protein, 9 aa, Homo sapiens
VMPICLPSK

SEQ ID NO: 11 - protein, 18 aa, Homo sapiens
AVGDKLPECEAVCGKPKN

SEQ ID NO: 12 - protein, 12 aa, Homo sapiens
DIAPTLTLYVGK

SEQ ID NO: 13 - protein, 12 aa, Homo sapiens
SCAVAEYGVYVK

SEQ ID NO: 14 - protein, 16 aa, Homo sapiens
ALYSGNDVTDISDDRF

SEQ ID NO: 15 - protein, 17 aa, Homo sapiens
NGYVEHLFRYQCKNYYR

SEQ ID NO: 16 - protein, 15 aa, Homo sapiens
NQVDIGLIKLKQKVL

SEQ ID NO: 17 - protein, 22 aa, Homo sapiens
NYAEVGRVGYVSGWGQSDNFKL

SEQ ID NO: 18 - protein, 18 aa, Homo sapiens
YDCITHYEGSTCPKWKAP

SEQ ID NO: 19 - protein, 5 aa, Homo sapiens
FCVGM

SEQ ID NO: 20 - protein, 7 aa, Homo sapiens
YAAGILS